(19) United States
(12) Patent Application Publication
(10) Pub. No.: US 2025/0082797 A1
(43) Pub. Date: Mar. 13, 2025

(54) Methods for In Vivo Tracking of Cells

(71) Applicant: Wisconsin Alumni Research Foundation, Madison, WI (US)

(72) Inventors: Reinier Hernandez, Madison, WI (US); Anatoly Pinchuk, Fitchburg, WI (US); Liudmila Lambert Lepesevich, Madison, WI (US); Zachary Rosenkrans, Madison, WI (US); Anna Thickens, Madison, WI (US); Nathan Clemons, Madison, WI (US)

(21) Appl. No.: 18/829,184

(22) Filed: Sep. 9, 2024

Related U.S. Application Data
(60) Provisional application No. 63/581,215, filed on Sep. 7, 2023.

(51) Int. Cl. A61K 51/12; A61K 51/04

(52) U.S. Cl. CPC A61K 51/1203; A61K 51/0497; A61K 2123/00

(57) ABSTRACT

Methods of radiolabeling of cell surface glycans for in vivo cell tracking and imaging as well as populations of radiolabeled cells and uses thereof are disclosed.

### CROSS-REFERENCE TO RELATED APPLICATIONS

This application claims priority to U.S. Provisional Application No. 63/581,215, filed on Sep. 7, 2023, which is incorporated herein by reference in its entirety.

### BACKGROUND OF THE DISCLOSURE

### Field of Invention

This disclosure relates to methods of direct radiolabeling on cell surface glycans for in vivo tracking and imaging.

### Technical Background

Macrophages and their progenitor monocytes—referred to herein as MΦ— are a heterogeneous population of immune cells implicated in a series of debilitating, often life-threatening conditions such as rheumatoid arthritis, atherosclerosis, acute ischemia, myocardial infarction, diabetes, graft versus host disease, and cancer (Chan and Viswanathan, 2019). MΦ, which are found in the body as both tissue-resident macrophages and as circulating monocytes, are involved in immune surveillance, inflammation, and tissue repair (Soehnlein and Lindbom, 2010). Depending upon their functional phenotype, MΦs can be classified as classically activated or pro-inflammatory M1, and alternatively, activated or inflammation-resolving M2 subtypes (Gordon, 2007, Sica and Mantovani, 2012, Murray et al., 2014). MΦ have incredible functional plasticity that allows them to change phenotype dynamically in response to local environmental cues. The loss of this plasticity results in the pathogenic alteration of the M1/M2 phenotype balance within diseased tissues. Restoring phenotypic balance between pro-inflammatory and anti-inflammatory tissue macrophages constitutes an appealing therapeutic strategy (Hutchinson, Riquelme et al. 2011, Fraser, Pass et al. 2017). Because circulating MΦs can infiltrate and repopulate target tissues, MΦs polarized ex vivo into classically or alternatively activated phenotypes through genetic engineering and culturing strategies can be administered systemically to achieve the desired balance at the disease site (Chan and Viswanathan, 2019, Bart et al., 2021). Early clinical experiences with MΦs cell therapies have shown promising results for treating inflammatory diseases and cancer, but challenges limit the widespread adoption of this modality.

Due to the myriad of disease-specific roles of MDs, several aspects must be taken into account when designing an exogenous MΦ therapy (Ardura, Rackov et al. 2019, Zhang, Yang et al. 2021), including the timing of MΦ administration, in vivo MΦ phenotypic/functional stability, MΦ's ability to infiltrate and reshape the infiltrating/resident cell populations, the capacity of the disease site to recruit and retain MΦ from circulation, as well as the method for ex vivo MΦ reprogramming and expansion. Several of these limitations can be effectively addressed by implementing imaging strategies to track MΦ trafficking in vivo.

Indeed, the ability to efficiently label cells, such as MΦ and other administered therapeutic cells for tracking in vivo is crucial for research and development of new therapeutic treatments. However, current methods for cell labeling are limited. For example, current methods for radiolabeling cells are largely inefficient, involve harsh conditions, and have poor stability.

Due to high detection sensitivity and signal penetration compared to other non-invasive imaging modalities, nuclear medicine imaging technologies (i.e., PET and SPECT) are best suited to track the distribution of the relatively low number of administered therapeutic cells in vivo (Bulte and Daldrup-Link 2018, Ashmore-Harris, Iafrate et al. 2020). These methodologies require either direct labeling of cells with radioactive isotopes or indirect labeling through the expression of a reporter gene encoding a protein or receptor that binds a radioactive molecule (radiotracer) with high affinity (Kiraga, Kucharzewska et al. 2021). Direct labeling methods are well suited for macrophage radiolabeling given their relative simplicity compared to the genetic engineering route and the limited in vivo proliferation of MDs, leading to negligible signal dilution due to cell division. Reported attempts to directly radiolabel macrophages for in vivo imaging have relied on the incorporation of radiotracer into cells via metabolic uptake (¹⁸F-FDG), passive diffusion (¹¹¹In/⁸⁹Zr-oxine), or phagocytosis of ¹⁸F-labelled nanoparticles (Ritchie, Mileshkin et al. 2007, Jeong, Yoo et al. 2019, Sato, Stringaris et al. 2020).

However, these nonspecific labeling methods suffer from low activity incorporation yields and poor in vivo label stability which limits the number of cells that can be tracked and for how long. Thus, a need exists to develop efficient, biocompatible, and stable radiolabeling methods that enable longitudinal in vivo cell tracking. Developing such strategies presupposes a significant advancement in the inventors' ability to improve the accuracy, efficacy, and safety of cell therapies beyond therapeutic MΦ.

### SUMMARY OF THE DISCLOSURE

This disclosure relates to methods of direct radiolabeling of cell surface glycans for in vivo cell tracking and imaging and populations of radiolabeled cells and uses thereof.

In a first aspect, the present disclosure provides a method of labeling a cell for in vivo tracking. The method includes the steps of (a) generating an aldehyde group on a cell surface glycan of a cell via reaction with an oxidizing agent, and (b) reacting the aldehyde group on the cell surface glycan with an aldehyde-reactive bifunctional chelator. The bifunctional chelator reacts with the aldehyde group on the cell surface glycan to ligate the bifunctional chelator to the cell surface glycan to form a labeled cell.

In one embodiment of the first aspect, the bifunctional chelator is generated by conjugating an aldehyde/ketone-reactive group to a chelator. In one embodiment of the first aspect, the aldehyde/ketone-reactive group is an aminooxy group. In one embodiment of the first aspect, the aldehyde/ketone-reactive group is a hydrazine group. In one embodiment of the first aspect, the chelator is deferoxamine. In one embodiment of the first aspect, the chelator is 1,4,7,10-tetraazacyclododecane-1,4,7-triacetic acid (DO3A) or one of its derivatives; 1,4,7-triazacyclononane-1,4-diacetic acid (NODA) or one of its derivatives; 1,4,7-triazacyclononane-1,4,7-triacetic acid (NOTA) or one of its derivatives; 1,4,7,10-tetraazacyclododecane-1,4,7,10-tetraacetic acid (DOTA) or one of its derivatives; 1,4,7-triazacyclononane,1-glutaric acid-4,7-diacetic acid (NODAGA) or one of its derivatives; 1,4,7,10-tetraazacyclodecane,1-glutaric acid-4,7,10-triacetic acid (DOTAGA) or one of its derivatives; 1,4,8,11-tetraazacyclotetradecane-1,4,8,11-tetraacetic acid (TETA) or one of its derivatives; 1,4,8,11-tetraazabicyclo[6.6.2]hexadecane-4,11-diacetic acid (CB-TE2A) or one of its derivatives; diethylene triamine pentaacetic acid (DTPA), or its diester, or one of its derivatives; 2-cyclohexyl diethylene triamine pentaacetic acid (CHX-A″-DTPA) or one of its derivatives; 2-(4-Isothicyanatobenzyl)-1,2,7,10,13-hexaazacyclooctadecane-1,4,7,10,13,16-hexaacetic acid (HEHA) or one of its derivatives; deferoxamine (DFO) or one of its derivatives; 1,2-[[6-carboxypyridin-2-yl]methylamino]ethane (H2dedpa) or one of its derivatives; HOPO or one of its derivatives; MACROPA or one of its derivatives; a CROWN ethers or one of its derivatives; DADA or one of its derivatives.

In one embodiment of the first aspect, the bifunctional chelator is an aminooxy-deferoxamine. In one embodiment of the first aspect, the oxidizing agent is NaIO₄. In one embodiment of the first aspect, the radionuclide is conjugated to the bifunctional chelator. In one embodiment of the first aspect, the method further includes conjugating a radionuclide to the ligated bifunctional chelator. In one embodiment of the first aspect, prior to ligation to the cell surface glycan, the bifunctional chelator is incubated with a radionuclide to generate a radionuclide conjugated bifunctional chelator complex. In one embodiment, the radionuclide is ⁸⁹Zr, ⁶⁴Cu, ⁶¹Cu, ⁵²Mn, ⁵¹Mn, ⁸⁶Y, ¹¹¹In ¹⁷⁷Lu, ¹⁵³Sm, ⁹⁹mTc, ⁶⁸Ga, ⁶⁶Ga, ⁶⁷Ga, ^{44/43}Sc, ⁴⁵Ti, ⁵⁵Co, ¹⁸F, ¹⁶¹Tb, or ¹⁵²Tb.

In one embodiment of the first aspect, the cell is a macrophage, a T-cell, a B-cell, a natural killer cell, a monocyte, a granulocyte, a neutrophil, a dendritic cell, a neutrophil, a fibroblast, a mesenchymal stromal cell, or a peripheral blood mononuclear cell.

In a second aspect, the present disclosure provides a method of generating a population of radiolabeled cells for in vivo tracking. The method includes the steps of (a) generating aldehyde groups on cell surface glycans of a population of cells via reaction with an oxidizing agent; (b) reacting the aldehyde groups on the cell surface glycans with an aldehyde-reactive bifunctional chelator to ligate the bifunctional chelator to the cell surface glycans; and (c) conjugating a radionuclide to the bifunctional chelator to generate a radionuclide labeled cell population.

In one embodiment of the second aspect, the viability of the cell viability is greater than about 90% after step (a).

In a third aspect, the present disclosure provides a method of tracking and imaging a radiolabeled cell in vivo. The method includes (a) administering the radionuclide labeled cell of the second aspect to a subject; and (b) tracking and imaging the radiolabeled cell in the subject by PET/CT or SPECT/CT or Planar scan of the subject.

In one embodiment of the third aspect, the radionuclide labeled cell is administered intravenously, intraperitoneally, subcutaneously, intrathecally, or intracranially.

In a fourth aspect, the present disclosure provides a radionuclide labeled cell that includes a cell surface glycan ligated with a bifunctional chelator. A radionuclide is conjugated to the bifunctional chelator.

In one embodiment of the fourth aspect, the cell surface glycan is a sialic acid. In one embodiment of the fourth aspect, the bifunctional chelator comprises an aminooxy group or a hydrazine group conjugated to a chelator. In one embodiment of the fourth aspect, the chelator is deferoxamine, 1,4,7,10-tetraazacyclododecane-1,4,7-triacetic acid (DO3A) or one of its derivatives; 1,4,7-triazacyclononane-1,4-diacetic acid (NODA) or one of its derivatives; 1,4,7-triazacyclononane-1,4,7-triacetic acid (NOTA) or one of its derivatives; 1,4,7,10-tetraazacyclododecane-1,4,7,10-tetraacetic acid (DOTA) or one of its derivatives; 1,4,7-triazacyclononane,1-glutaric acid-4,7-diacetic acid (NODAGA) or one of its derivatives; 1,4,7,10-tetraazacyclodecane,1-glutaric acid-4,7,10-triacetic acid (DOTAGA) or one of its derivatives; 1,4,8,11-tetraazacyclotetradecane-1,4,8,11-tetraacetic acid (TETA) or one of its derivatives; 1,4,8,11-tetraazabicyclo[6.6.2]hexadecane-4,11-diacetic acid (CB-TE2A) or one of its derivatives; diethylene triamine pentaacetic acid (DTPA), or its diester, or one of its derivatives; 2-cyclohexyl diethylene triamine pentaacetic acid (CHX-A″-DTPA) or one of its derivatives; 2-(4-Isothicyanatobenzyl)-1,2,7,10,13-hexaazacyclooctadecane-1,4,7,10,13,16-hexaacetic acid (HEHA) or one of its derivatives; deferoxamine (DFO) or one of its derivatives; 1,2-[[6-carboxypyridin-2-yl]methylamino]ethane (H2dedpa) or one of its derivatives; HOPO or one of its derivatives; MACROPA or one of its derivative; a CROWN ether or one of its derivative; or DADA or one of its derivatives.

In one embodiment of the fourth aspect, the radionuclide is ⁸⁹Zr, 64Cu, ⁶¹Cu, ⁵²Mn, ⁵¹Mn, ⁸⁶Y, ¹¹¹In, ¹⁷⁷Lu, ¹⁵³Sm, ⁹⁹mTc, ⁶⁸Ga, ⁶⁶Ga, ⁶⁷Ga, ^{44/43}Sc, ⁴⁵Ti, ⁵⁵Co, ¹⁸F, ¹⁶¹Tb, or ¹⁵²Tb.

These and other features and advantages of the present invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF FIGURES

The accompanying drawings, which are included to provide a further understanding of the disclosure, are incorporated in and constitute a part of this specification, illustrate embodiments of the disclosure, and together with the detailed description serve to explain the principles of the disclosure.

FIG. 1. Clinical Trials of Cell Therapies. Overview of cell therapies under clinical development.

FIG. 2. Preclinical and clinical imaging techniques. Examples of preclinical and clinical imaging techniques for in vivo cell imaging using ex vivo cell radiolabeling techniques.

FIG. 3. Schematic of a direct cell labeling workflow. General workflow for the isolation, radiolabeling, administration, and in vivo nuclear medicine imaging of the labeled cells.

FIGS. 4A-4B. FIG. 4A is a schematic of direct conjugation method for conjugating a deferoxamine (DFO) chelator to surface proteins using amide bond ligation. FIG. 4B shows longitudinal PET imaging of mice injected with approximately 5 million MΦ labeled with ⁸⁹Zr (according to FIG. 4A) up to six days after injection.

FIG. 5. Table summarizing reported parameters for the current ex vivo radiolabeling methodologies in terms of number, radiolabeling efficiency, and viability of administered cells.

FIG. 6. Schematic showing glycan ligation. Cell surface glycans be conjugated with bifunctional chelators. For example, ligation can occur by generation of cell surface aldehydes by mild NaIO₄ oxidation of sialic acid and subsequent oxime ligation with Aminooxy-DFO.

FIGS. 7A-7C. FIG. 7A shows synthesis of an aldehyde/ketone reactive bifunctional chelator based on deferoxamine (DFO). DFO was derivatized with aminooxy acetic acid, and aminooxy-DFO (AOD) was purified by flash chromatography (>95%) and identified by NMR and mass spectrometry. Additional bifunctional chelators can be generated based on other chelators disclosed herein. FIG. 7B shows purity and identity of AOD compound as measured by mass spectrometry. FIG. 7C shows the ¹H-NMR confirming AOD's structure.

FIG. 8. Optimization of oxidation conditions. Glycan oxidation conditions using cell line U937 were optimized by varying the time, temperature, and sodium periodate concentrations. 1 mM oxidation for up to 1 hour did not affect viability significantly which remained over 90%. Incubation at 4° C. allowed for longer oxidation times. One hour oxidation was shown to yield good conjugation yields.

FIGS. 9A-9D. Radiolabeling of immortalized U937 monocytes (Mos). Mos were suspended in 1 mL PBS (pH 7.4) and oxidized with NaIO₄ (1 mM) for 20 min. Upon washing and resuspension into 1 mL PBS (pH 6.5), the cells were incubated with AOD (200 μM) in aniline (10 mM) for 40 min. Once conjugated, the cells (2-5×10⁶) were radiolabeled with ⁸⁹Zr (1 mCi, 37 MBq) for 40 min and centrifuged to remove unconjugated ⁸⁹Zr. FIG. 9A shows the radioTLC chromatograms of (FIG. 9B) free ⁸⁹Zr, the radiolabeling reaction mixture (FIG. 9C), and (FIG. 9D) the purified radiolabeled cells.

FIGS. 10A-10C. Summary of the purity, radiochemical yield, and cell viability for the radiolabeling of cells using surface glycan conjugations. FIG. 10A shows the parameters for the different sources of monocytes. FIG. 10B shows RadioTLC determines radiochemical purity and yields. FIG. 10C shows chromatogram and analysis of the radioTLC displaying quantitative information.

FIGS. 11A-11C. PET/CT of U937 Monocyte Cell Line. NSG mice were injected with 126-150 μCi (3.5-4.1×10⁵ cells/injection). FIG. 11A shows longitudinal PET/CT scans that were acquired over a 7 day period. FIG. 11B shows that most of the activity was localized to the liver and spleen. FIG. 11C shows ex vivo biodistribution performed at day 8 to provide more accurate tissue distribution and confirm PET/CT.

FIGS. 12A-12C. PET/CT Imaging of ⁸⁹Zr-PBMCs. NSG mice were injected with about 20 μCi (2.0-2.5×10⁵ cells/injection)⁸⁹Zr-labeled peripheral blood mononuclear cells (PBMCs). FIG. 12A shows longitudinal PET/CT scans acquired over a 8 day period. FIG. 12B shows that activity localization was predominantly in the lungs, liver, and spleen. FIG. 12C shows ex vivo biodistribution performed at day 8 to provide more accurate tissue distribution and confirm PET/CT.

FIGS. 13A-13C. Imaging iPSC-derived human/NHP ⁸⁹Zr-Macrophages. NSG mice were injected with about 25 μCi (0.15-2.5×10⁵ cells/injection)⁸⁹Zr-labeled macrophages derived from NHP and human iPSCs. FIG. 13A shows Longitudinal PET/CT scans acquired over a 7 day period. FIGS. 13B and 13C show that most of the activity was localized to the liver and spleen.

FIG. 14. Ex vivo biodistribution of ⁸⁹Zr-Macrophages. ⁸⁹Zr-Macrophages were intravenously injected in NSG mice and the animals were sacrificed at day 7 post injection, and the tissues collected, weighted and counter in a gamma counter.

FIGS. 15A-15C. Imaging iPSC-derived NHP ⁸⁹Zr-Macrophages. NSG mice were injected with about 134 μCi (1.44×10⁶ cells/injection)⁸⁹Zr-labeled macrophages derived from NHP. FIG. 15A shows Longitudinal PET/CT scans acquired over a 7 day period. FIG. 15B shows that most of the activity was localized to the liver and spleen. FIG. 15C shows ex vivo biodistribution performed at day 7 confirming that the main uptake was in the liver and the spleen.

FIG. 16. Radiolabeling of T cells. Comparison of radiochemical yield and radiochemical purity measured by radio-TLC between labeled Jurkat T cells and U937 monocytes. Similar results are observed in both cell types.

FIGS. 17A-17C. Imaging ⁸⁹Zr-Jurkat human T cells. One NSG mouse was injected with 69 μCi (5.18×10⁶ cells/injection) of ⁸⁹Zr-labeled Jurkat T cells. FIG. 17A shows Longitudinal PET/CT scans of ⁸⁹Zr-Jurkat T cells acquired over a 7 day period, with strong liver and bone signal at all time points. FIG. 17B shows that most of the activity was localized to the liver and spleen based on ROI analysis. FIG. 17C shows ex vivo biodistribution performed at day 7 confirming that the main uptake was in the liver and lungs. Interestingly significant bone marrow localization of the labeled cells was observed.

FIGS. 18A-18D. Radiolabeling of iPSC derived NHP neutrophils. FIG. 18A shows the chromatograms of free ⁸⁹Zr. FIG. 18B shows the chromatogram of the radiolabeling reaction mixture. FIG. 18C shows the chromatogram of the purified radiolabeled cells. FIG. 18D shows the autoradiograph of the radio-TLC.

FIGS. 19A-19C. Imaging iPSC derived NHP ⁸⁹Zr- neutrophils. NSG mice were injected with ⁸⁹Zr-labeled neutrophils. FIG. 19A shows Longitudinal PET/CT scans acquired over a 7 day period. FIG. 19B shows that most of the activity was localized to the liver and spleen. FIG. 19C shows ex vivo biodistribution performed at day 7 confirming that the main uptake was in the liver and spleen.

FIG. 20. Treatment with clodronate liposomes alters the distribution of ⁸⁹Zr-U937 cells. NSG mice were treated with phagocytic cell-depleting clodronate liposomes 24h before or after administration of ⁸⁹Zr-U937 cells. Treatment with control liposomes did not affect the typical distribution of ⁸⁹Zr-U937 cells to the liver and spleen of NSG mice (top panel). Significant ⁸⁹Zr-U937 spleen accumulation was observed in mice receiving clodronate 24 h before (middle panel). On the other hand, post-treatment with clodronate resulted in a marked redistribution of the liver signal to the bone marrow (bottom panel).

FIG. 21A-21D. Quantitative analysis of PET/CT images and ex vivo biodistribution. FIG. 21A shows treatment with control liposomes does not affect the distribution of ⁸⁹Zr-U937 cells. Treatment with clodronate liposomes 24 h before (FIG. 21B) or after (FIG. 21C)⁸⁹Zr-U937 injection resulted in marked difference in liver, spleen, and bone marrow, suggesting the viability of the injected ⁸⁹Zr-U937 cell in vivo. FIG. 21D shows ex vivo biodistribution performed at day 7 confirming the trends observed with the imaging. Interestingly, the whole-body uptake of ⁸⁹Zr-U937 remained relatively unchanged between treatments.

FIG. 22. RadioTLC of NHP ⁸⁹Zr-monocytes for primate imaging studies. Excellent radiochemical purities >93% were obtained for ⁸⁹Zr-monocytes.

FIGS. 23A-23B. Longitudinal PET/CT of iPSC derived NHP ⁸⁹Zr-Monocytes in Mauritius Macaques. FIG. 23A shows Mauritius macaques received ˜300 μCi of ⁸⁹Zr-Monocytes (2.1×10⁶ cells) and longitudinal PET/CT scans acquired. Initial lung signal was observed at 30 min, with subsequent redistribution to liver and spleen of the animals. Persistence of PET signal up to 7 days evidenced the in vivo stability of the ⁸⁹Zr radiolabeled using the novel protocol. Subject one received autologous cells while subject 2 received allogeneic cells. FIG. 23B shows ROI analysis of PET/CT scans showing prominent ⁸⁹Zr-Monocytes uptake and prolonged retention in the liver and spleen in both subjects. ROI analysis trends of ⁸⁹Zr-Monocyte in Mauritius macaque, with heightened bone marrow uptake visible.

FIGS. 24A-24B. The high radiolabeling efficiency of the novel protocol allows for detection of small cell clusters. FIG. 24A shows the ability to detect a small concentration of labeled ⁸⁹Zr-Monocytes in the bone marrow and other organs like the lung.

FIG. 24B shows the very low number of circulating ⁸⁹Zr-Monocyte in blood.

### DETAILED DESCRIPTION OF THE DISCLOSURE

It is to be understood that the particular aspects of the specification are described herein are not limited to specific embodiments presented and can vary. It also will be understood that the terminology used herein is for the purpose of describing particular aspects only and, unless specifically defined herein, is not intended to be limiting. Moreover, particular embodiments and examples disclosed herein can be combined with other embodiments and examples disclosed herein, as would be recognized by a skilled person, without limitation.

All publications, patents, and patent applications cited herein are hereby expressly incorporated by reference in their entirety for all purposes.

Throughout this specification, unless the context specifically indicates otherwise, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an,” and “the” include plural referents unless the context clearly indicates otherwise.

Unless otherwise indicated or otherwise evident from the context and understanding of one of ordinary skill in the art, values herein that are expressed as ranges can assume any specific value or sub-range within the stated ranges in different embodiments of the disclosure, to the tenth of the unit of the lower limit of the range, unless the context clearly dictates otherwise.

As used herein, ranges and amounts can be expressed as “about” a particular value or range. About also includes the exact amount. For example, “about 5%” means “about 5%” and also “5%.” The term “about” can also refer to ±10% of a given value or range of values. Therefore, about 5% also means 4.5%-5.5%, for example.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.

As used herein, the term “subject” refers to a patient in need of a treatment thereof or an animal. For example, the patient, subject, or individual can be a mammal, such as a human to be treated for a disorder, condition, or a disease.

### Overview

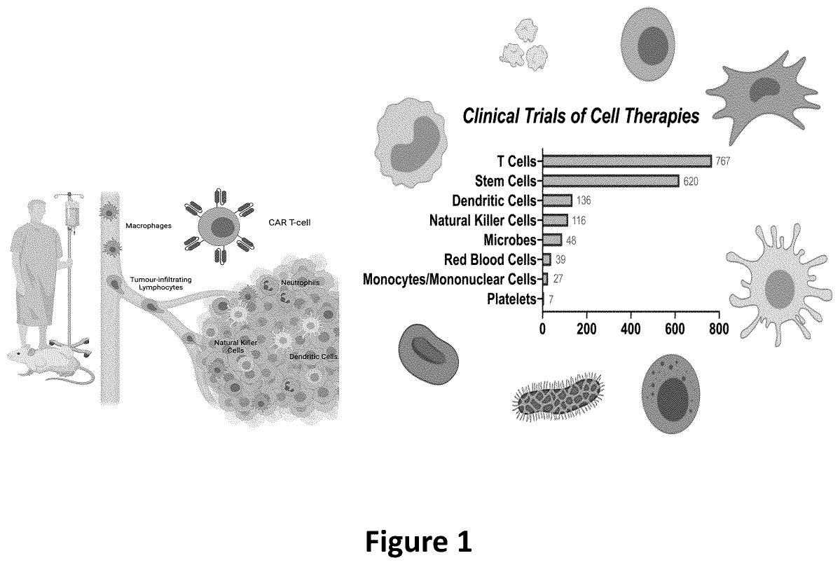

The present disclosure is directed to methods for radiolabeling cells for in vivo tracking by direct conjugation of radiolabels to cell surface glycans and populations of and therapeutic compositions comprising such radiolabeled cells. Without wishing to be bound by theory, it is believed that the labeling strategies contemplated herein can be applied to cell-based therapies (e.g., as shown in FIG. 1), including stem cell-based, chimeric antigen receptor (CAR)-based, and other non-stem cell based therapies to advance preclinical and clinical development of these cell therapies by allowing noninvasive imaging technologies to inform patient selection, assess homing to target tissues, predict treatment response, and evaluate potential life-threatening toxicities.

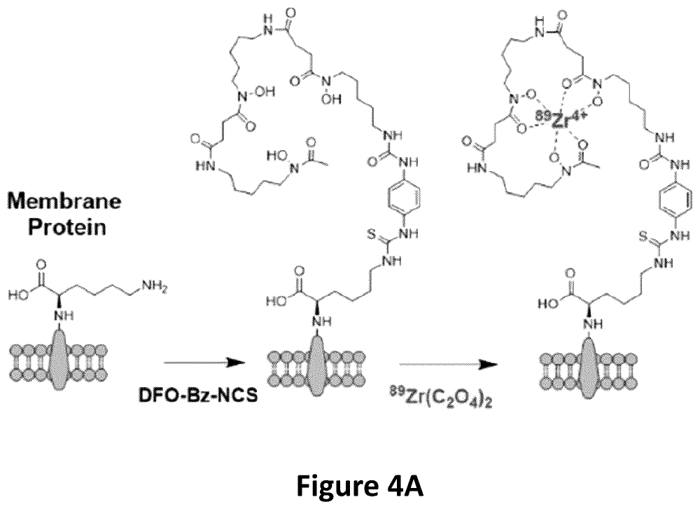

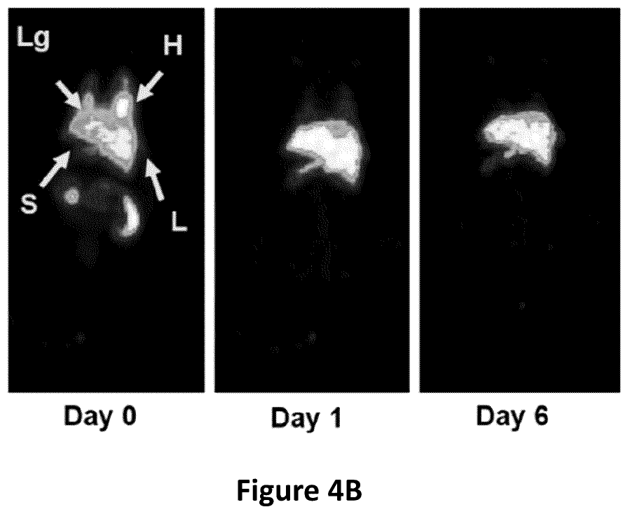

As disclosed herein, cell surface glycans can be oxidized and ligated via an oxime ligation reaction to a chelator, e.g., deferoxamine (DFO), which enables an efficient and stable capture of radionuclides, such as, for example, ⁸⁹Zr, to cell surfaces. An alternative strategy is to chemically anchor radioisotopes via amide or maleimide linkages to proteins in the cell membrane of cells (FIG. 4A). Covalent binding to proteins improves the stability of the cell label compared to non-specific oxine-based labeling by limiting the dissociation of the radioisotopes in vivo.

As disclosed herein, a direct protein labeling route has been successfully employed to radiolabel MΦ with ⁸⁹Zr for longitudinal PET imaging in mice. Using a previously reported method relying on thiourea ligations, the bifunctional chelator p-SCN-Bn-deferoxamine was conjugated to the lysine residues of surface proteins in MΦ isolated from human peripheral blood (Bansal, Pandey et al. 2015, Bansal, Pandey et al. 2020). This labeling approach required the pre-labeling of p-SCN-Bn-deferoxamine with ⁸⁹Zr in aqueous media before incubation with the cells, which resulted in significant hydrolysis of the isothiocyanate reactive group in the bifunctional chelator and poor overall incorporation in cells. Although an exceptional in vivo radiolabel stability was observed with this approach, radiochemical yields remained comparable to previously reported methods. Additionally, chemically modifying cell surface proteins may impact the viability and function of therapeutic cells.

The presently disclosed methods significantly improve radiolabeling paradigms for macrophages (and other cell types, such as T-cells, B-cells, natural killer cells, monocytes, granulocytes, dendritic cells, mesenchymal stromal cells, neutrophils, fibroblasts, peripheral blood mononuclear cells, and including engineered CAR versions of these cells, and other cell types) tracking by chemically anchoring the radionuclide to cell surface glycans. Cell surface glycans are branched polysaccharides involved with cellular structure, cell-cell communication, immunity, and cancer. Glycans are ubiquitously found in cell membrane components, including glycosphingolipids, proteoglycans, and O-link and N-linked glycoproteins (Cheng, Tang et al. 2021). This relative abundance makes glycans attractive anchoring points for chemical conjugations (Zeng, Ramya et al. 2009). For example, oxidation of the vicinal diol in sialic acid can serve as an advantageous ligation target.

Additionally, chemical alterations to polysaccharide chains are less likely to have a survival or functional impact on the cell than protein-based modifications. The low yield of current direct cell labeling strategies can be addressed by conjugating a chelator (e.g., DFO) via an oxime ligation to sialic acid. This conjugation which can proceed under mild conditions enables the efficient and stable radiolabeling to live-cell with the positron-emitting radionuclide ⁸⁹Zr (Dirksen and Dawson 2008). In addition to targeting sialic acids for ligation, additional glycans are also contemplated herein, as well as glycan-specific chemistries that can be used for their ligation.

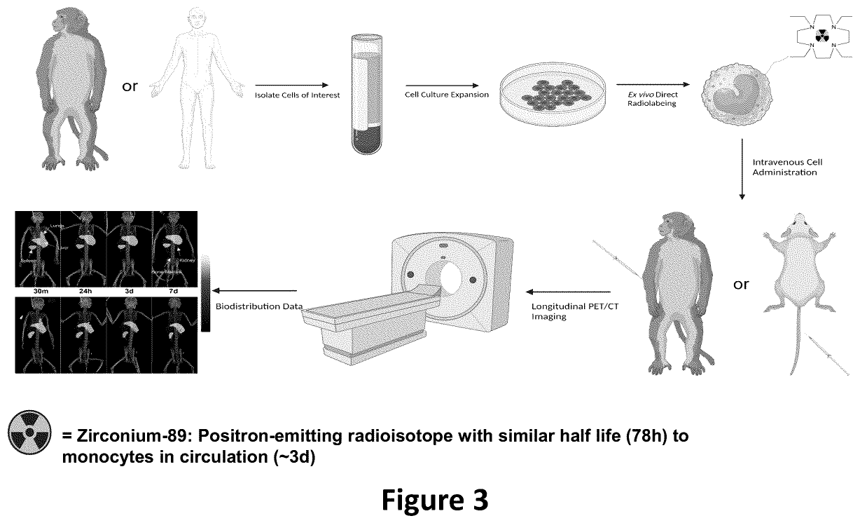

In one embodiment, the disclosed methods can be used to radiolabel human and/or non-human primates cells (e.g., MΦ) derived from peripheral blood (see FIG. 3) or induced pluripotent stem cells (iPSCs). In vivo positron emission tomography (PET/CT) imaging can be employed in non-human primates using radiolabeled MΦ to demonstrate the in vivo stability of the radiolabeled cells and the sensitivity of PET to detect relatively small numbers of injected cells. Such experiments are envisioned to demonstrate the biodistribution of systemically administered MΦ in non-human primates.

Additionally, this cell labeling method can help answer a standing question in the field regarding the equivalency, or lack thereof, of the in vivo behavior of MΦ derived from peripheral blood and those derived from more scalable iPSC sources. Finally, because of the anatomical and physiological similarities between human and non-human primates, the methods and results disclosed herein can have a direct bearing on the translation of monocytic therapies in humans.

### Generation of an Aldehyde Group on Cell Surface Glycans

Because different cell types may exhibit varying levels of surface glycans, in some embodiments, in some embodiments, cells can be supplemented with sialic acid analogs such as 9-deoxy-9-N-carboxybenzaldehyde-NeuAc (BA-NeuAc) or others.

In some embodiments, the oxidizing agent used to generate a reactive aldehyde group on the cell surface's glycan is NaIO₄. For example, in some embodiments, cells are incubated with an increasing concentration of NaIO₄ ranges from about 5 to about 500 μM. The duration can range from about 15 to about 120 minutes.

Additional oxidation agents are contemplated herein including, for example, pyridoxal 5′-phosphate. In addition, oxidation via use of glyoxylate, copper (II) salts, 1 M pyridine, and 1 M acetic acid is possible.

In some embodiments, the oxime ligation occurs with a catalyst, such as aniline, p-Methoxy aniline, 5-Methoxy anthranilic acid, m-Phenylenediamine, and/or p-Phenylenediamine. In some embodiments, the oxidation reaction occurs without a catalyst.

### Generation of an Aldehyde-Reactive Bifunctional Chelator

As used herein, the term “chelator” refers to a chemical agent that has a metal binding moiety function. A “bifunctional chelator” refers to a chemical agent that has a metal binding moiety function and a chemically reactive functional group. For example, chemically reactive functional groups can be chemically reactive with aldehydes and/or ketones.

A list of contemplated chelators further includes, but is not limited to, 1,4,7,10-tetraazacyclododecane-1,4,7-triacetic acid (DO3A) and its derivatives; 1,4,7-triazacyclononane-1,4-diacetic acid (NODA) or one of its derivatives; 1,4,7-triazacyclononane-1,4,7-triacetic acid (NOTA) or one of its derivatives; 1,4,7,10-tetraazacyclododecane-1,4,7,10-tetraacetic acid (DOTA) or one of its derivatives; 1,4,7-triazacyclononane,1-glutaric acid-4,7-diacetic acid (NODAGA) or one of its derivatives; 1,4,7,10-tetraazacyclodecane,1-glutaric acid-4,7,10-triacetic acid (DOTAGA) or one of its derivatives; 1,4,8,11-tetraazacyclotetradecane-1,4,8,11-tetraacetic acid (TETA) or one of its derivatives; 1,4,8,11-tetraazabicyclo[6.6.2]hexadecane-4,11-diacetic acid (CB-TE2A) or one of its derivatives; diethylene triamine pentaacetic acid (DTPA), its diester, or one of its derivatives; 2-cyclohexyl diethylene triamine pentaacetic acid (CHX-A″-DTPA) or one of its derivatives; 2-(4-Isothicyanatobenzyl)-1,2,7,10,13-hexaazacyclooctadecane-1,4,7,10,13,16-hexaacetic acid (HEHA) or one of its derivatives; deforoxamine (DFO) or one of its derivatives; 1,2-[[6-carboxypyridin-2-yl]methylamino]ethane (H2dedpa) or one of its derivatives; HOPO or one of its derivatives; MACROPA or one of its derivatives; a CROWN ether or one of its derivatives; or DADA or one of its derivatives.

In additional to the chemical reactions utilized herein, enzymatic pretreatment of cells, such as with the enzyme galactose oxidase, can catalyze formation of terminal galactose and N-acetylgalactosamine residues that can be targeted for bifunctional chelator ligation. In some embodiments, the aldehyde reactive group to react with aldehydes (and/or ketones) on cell glycans forming oxime ligations or linkages is an aminooxy group. In some embodiments, N-Boc-aminooxy acetic acid N-hydroxysuccinimide ester is used as an aminooxy group donor or a substrate. In some embodiments, it is contemplated to use hydrazine reactive moieties to form hydrazone linkages (e.g., a hydrazine-deferoxamine). Additional examples of linkage chemistries envisioned herein are described in Kömel et al. (“Oximes and Hydrazones in Bioconjugation: Mechanism and Catalysis,” Chem. Rev. 2017, 117, 10358-10376), which is incorporated by reference herein in its entirety.

In some embodiments, an aldehyde/ketone-reactive bifunctional chelator is incubated with a radionuclide. In some embodiments, a one-pot reaction is contemplated herein, wherein an oxidized cell is incubated with an aldehyde/ketone-reactive bifunctional chelator and subsequently labeled with a radionuclide.

In some embodiments, the radionuclide is ⁸⁹Zr. In some embodiments, other radionuclides suitable for PET/CT scan are contemplated herein such as ⁶⁴Cu, ⁶¹Cu, ⁵²Mn, ⁵¹Mn, ⁸⁶Y ¹¹¹In ¹⁷⁷Lu, ¹⁵³Sm, ⁹⁹mTc, ⁶⁸Ga, ⁶⁶Ga, ⁶⁷Ga, ^{44/43}Sc, ⁴⁵Ti, ⁵⁵Co, ¹⁸F, ¹⁶¹Tb, or ¹⁵²Tb, or other radiometals for nuclear imaging.

### Administration of a Radiolabeled Cell to a Subject and In Vivo Tracking by PET/CT Scanning

Radiolabeled cells generated by the disclosed methods can be formulated with a suitable carrier such as phosphate-buffered saline, Hank's buffered salt solution (HBSS), and/or other physiologically acceptable carriers.

In some embodiments, radiolabeled cells can be administered intravenously into a subject but other administration routes are contemplated herein, such as, intraperitoneally, subcutaneously, intrathecally, intracranially, and by other suitable routes.

Once administered to a subject, the radiolabeled cells can be tracked in vivo by PET/CT, SPECT/CT, and other nuclear medicine technologies known in the art.

### Additional Commercial Applications

In some embodiments, radiolabeled cells of the present disclosure may be employed in quality control for manufacturing cellular therapies.

In some embodiments, it is envisioned that the presently disclosed labeling method can be used during preclinical and clinical development of new cellular therapies through monitoring location and clearance of radiolabeled cells applied to a subject in a clinical setting.

In some embodiments, radiolabeled cells of the present disclosure can be used for diagnostic and/or therapeutic purposes where radiolabeled cells collect at sites in vivo that correspond to a particular disease state (e.g., a metastatic tumor, an infarct, an internal lesion, and/or the tumor microenvironment).

### Examples

### Example 1: Identification of the Conditions for Efficient, Biocompatible ⁸⁹Zr Labeling of Monocytes

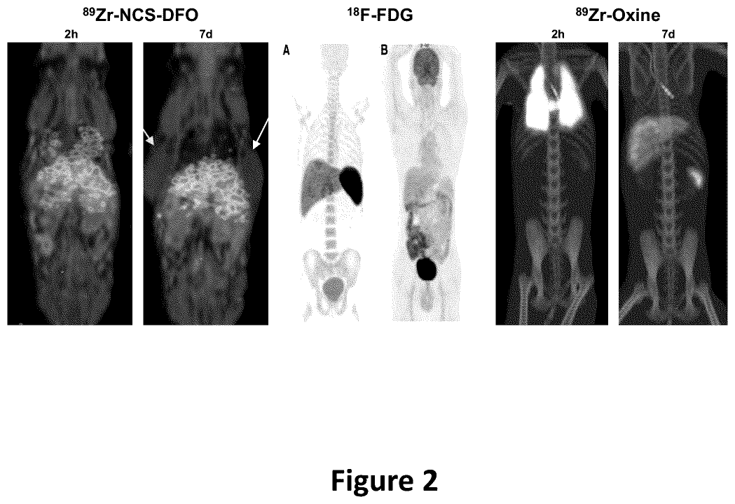

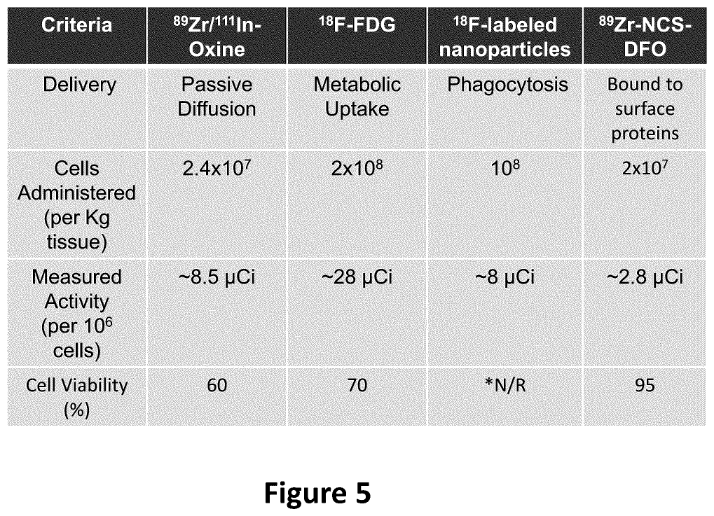

Introduction: Due to a relatively long half-life of 78.4 hours, which matches that of monocytes in circulation (˜3 days), ⁸⁹Zr is an ideal positron-emitter to track monocyte distribution by PET/CT (see FIG. 3). In preliminary studies, direct radiolabeling on cell surface proteins as described in FIG. 4A was achieved in human monocytes with ⁸⁹Zr. The ⁸⁹Zr-MΦ(˜200 million/kg) were administered to immunodeficient NSG mice and PET scans were acquired at days 0, 1, and 6 post-injection of the radiolabeled cells. FIG. 4B shows ⁸⁹Zr-MΦ initially homing to the lungs, heart, and liver. Heart and lung signals declined over time, indicating a significant redistribution of the cells from these organs into the liver and spleen. Indications of cell targeting to bone marrow were observed, although inconclusively, due to the low amounts of PET signal detected in these tissues. These data demonstrated the feasibility of direct conjugation methods to track MΦ distribution to organs with significant cell uptake, but due to low radioactivity incorporation per cell (˜2-5 μi/10\hat{ }6 cells), detection of a small number of infiltrating cells was challenging. These results were comparable with reported direct radiolabeling approaches that provide similar radioactivity levels per cell (Bansal, Pandey et al. 2015). Thus, the current direct labeling method (FIG. 2) offers limited utility in cases where the therapeutic effect depends on the infiltration of a relatively small number of cells, as in CAR-T therapies targeting solid tumors or HIV reservoirs in the brain. FIG. 5 summarizes reported parameters for the current ex vivo radiolabeling methodologies in terms of number, radiolabeling efficiency, and viability of administered cells.

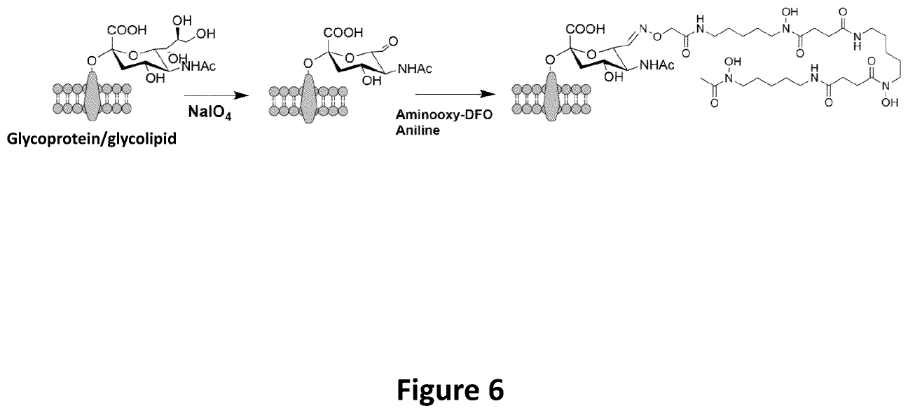

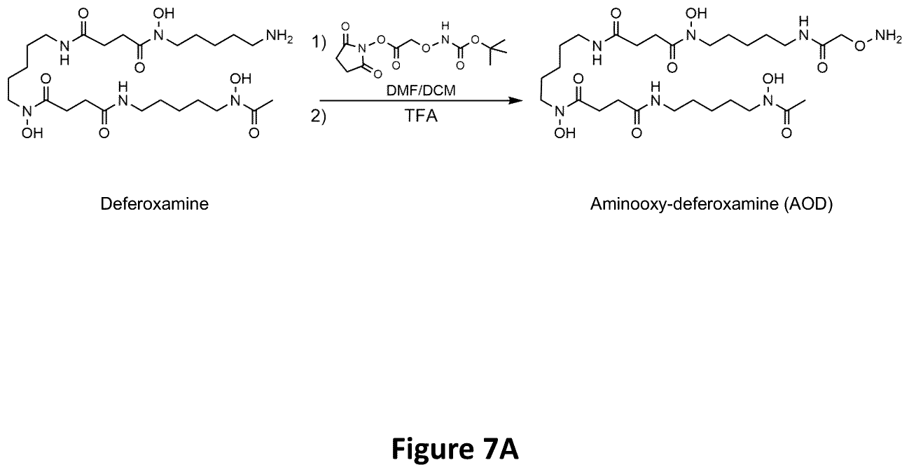

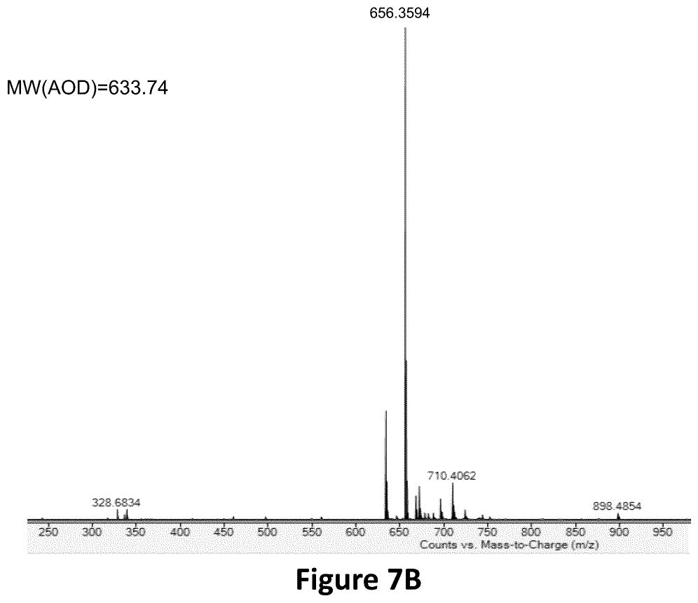

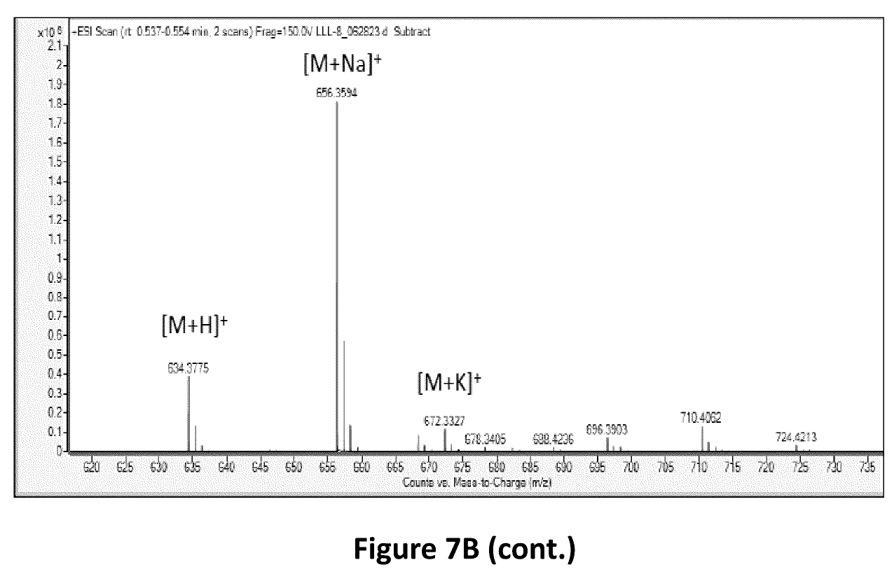

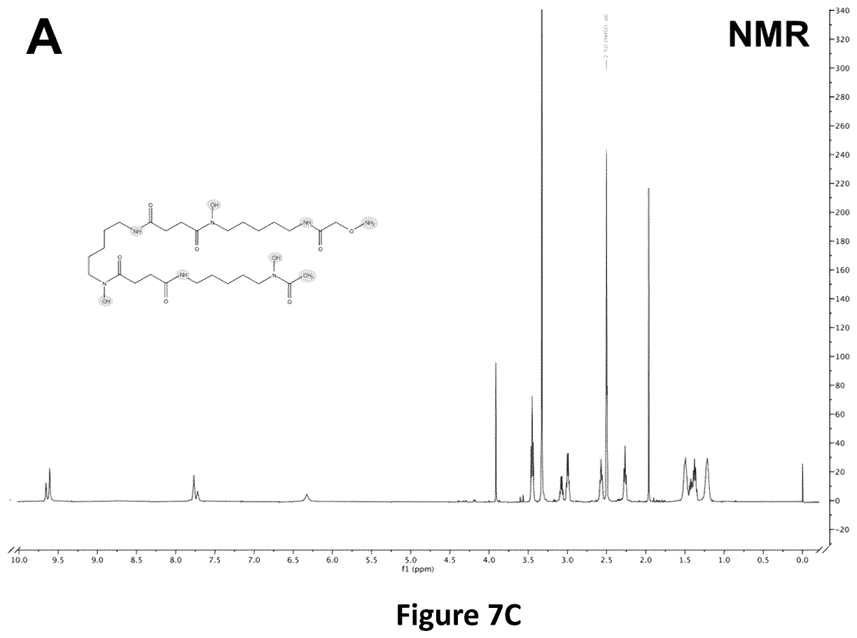

For this reason, an approach of introducing the aldehyde/ketone-reactive group to the chelator DFO was utilized to enable conjugation to the cell surface glycan sialic acid. This approach relies on the reaction of aminooxy reactive groups with aldehydes and ketones to form oxime linkages. Aldehydes can be readily and selectively generated by mild biocompatible periodate oxidation of membrane surface glycans containing sialic acid (Zeng, Ramya et al. 2009). Therefore, the objective was to synthesize an aldehyde-reactive bifunctional chelator (aminooxy-DFO) for conjugation to cell surface aldehydes and subsequent radiolabeling with ⁸⁹Zr. A schematic representation of the labeling approach is summarized in FIG. 6 and FIG. 7A.

Method 1: Synthesize glycan-reactive aminooxy-DFO intermediates. To a mixture of DFO mesylate and N,N-diisopropylethylamine (DIPEA) in dimethylformamide (DMF), an excess of N-Boc-aminooxy acetic acid N-hydroxysuccinimide ester was added. After an overnight reaction, the product was deprotected using TFA and purified by flash chromatography to yield aminooxy-DFO (AOD). The purity and identity of the compound was confirmed by NMR spectroscopy (FIG. 7C) and mass spectrometry (FIG. 7B). Radiolabeling of AOD with ⁸⁹Zr was carried out using the previously described methods. Briefly, 1-5 mCi of ⁸⁹Zr-oxalate was incubated with AOD (5 nmols/mCi) in 1M HEPES buffer (pH=7.5). ⁸⁹Zr-AOD was purified by solid-phase extraction using a reverse-phase cartridge and formulated in a biocompatible media and filtered through a 0.2 m sterile filter.

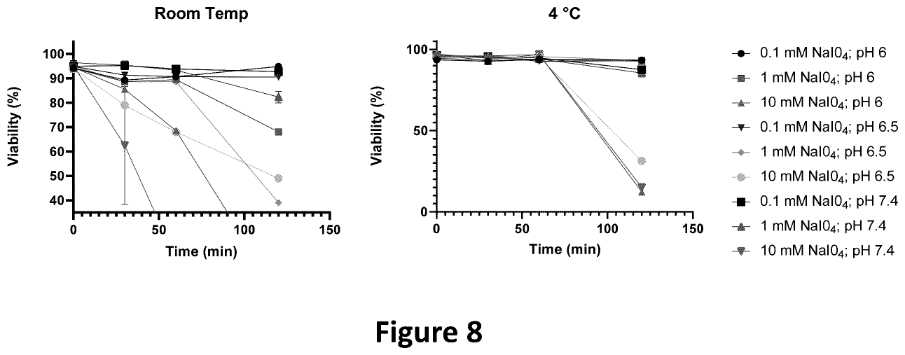

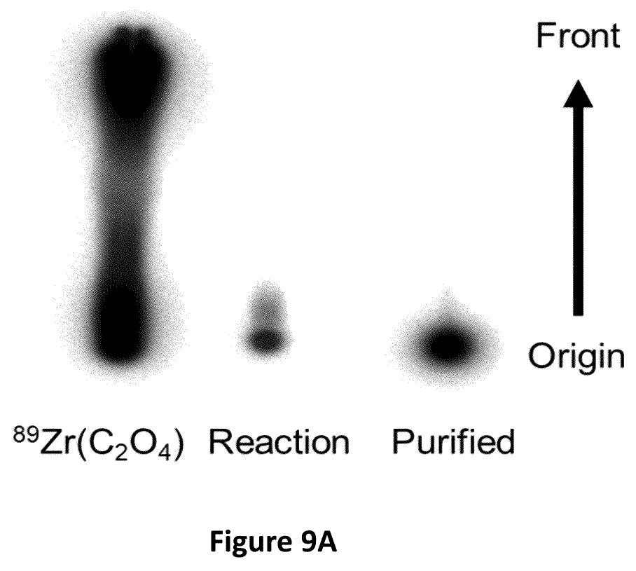

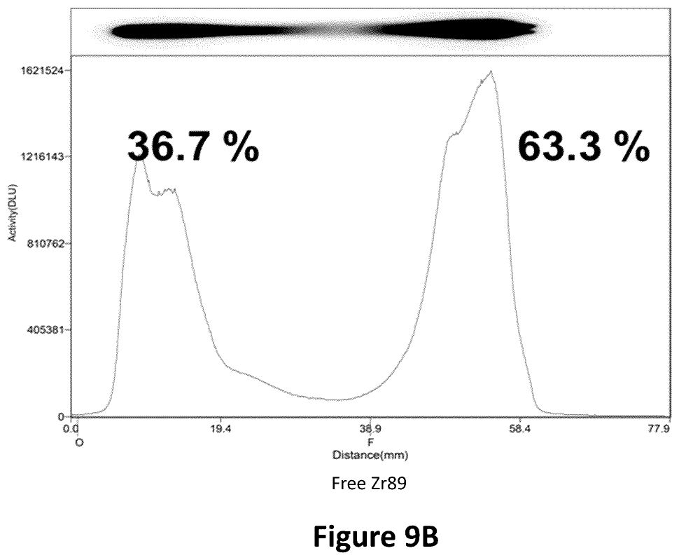

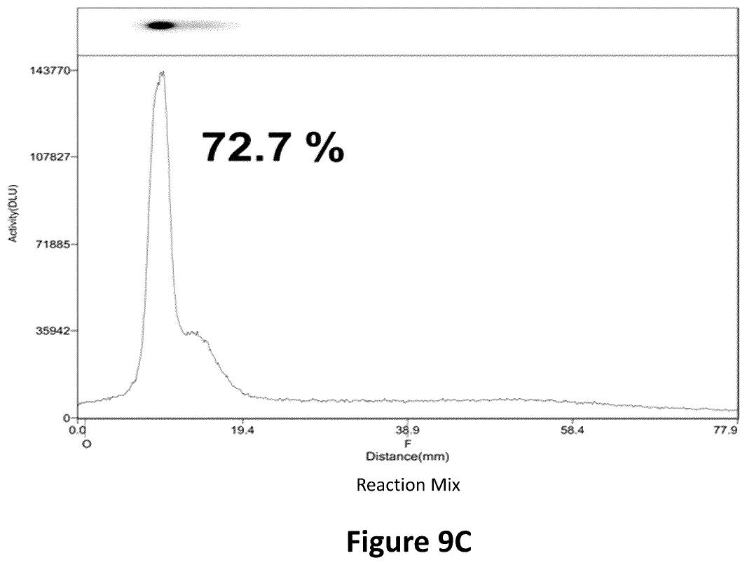

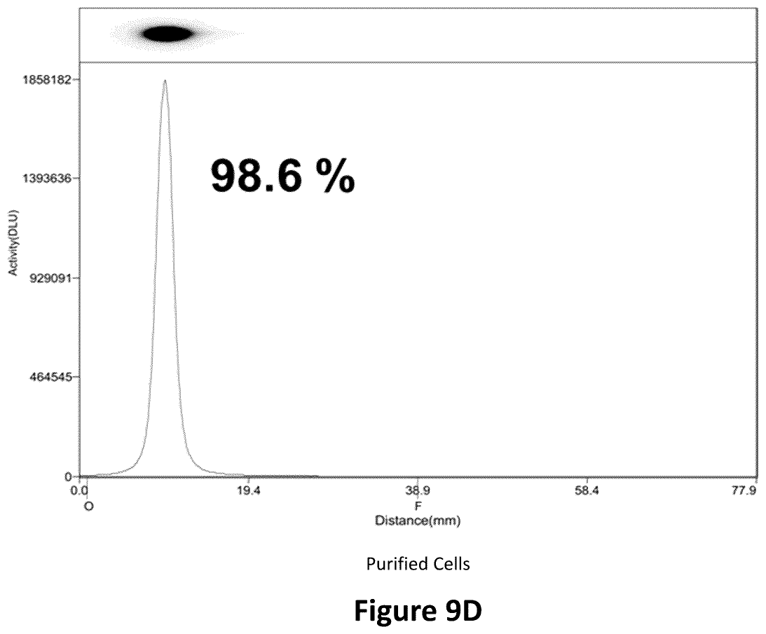

Method 2: Conditions for biocompatible oxime ligation and ⁸⁹Zr-labeling of monocytes. Reactive aldehydes were generated on the cell surface of the monocytic cell line U937 through sialic acid oxidation with NaIO₄. Oxidation conditions were optimized by varying NaIO₄ concentration (0.1-10 mM), pH (4.6-7.4), temperature (4 or 37° C.), and incubation time (15 min to 2 h). Subsequently, cells were incubated with AOD (100-500 μM) at room temperature for up to 1 h in the presence of aniline, and cells were washed and resuspended in PBS. Next, AOD-conjugated (5-7×10⁶) cells were radiolabeled with ⁸⁹Zr (37-74 MBq) at room temperature for 1h, then washed thrice to eliminate unlabeled ⁸⁹Zr, and the radiochemical yield was measured by TLC (FIG. 9A-9D). Cell viability was assessed at each step via trypan blue staining (FIG. 8).

Levels of radioactivity at about 100 μCi per million cells were consistently generated using the techniques disclosed herein. Further, as much as 500 μCi per million cells was not observed to be toxic to the cells (data not shown), and it is believed that higher levels of radioactivity can be used.

In alternative one-pot reactions, the cells were first incubated with AOD following the conditions mentioned above and subsequently radiolabeled with ⁸⁹Zr. Radiolabeled cells were centrifuged and washed with PBS to remove unincorporated radioactivity. Cell viability was recorded, and radiolabeling yields were determined as the fraction of cell-bound vs. added radioactivity. Additionally, the radio stability of ⁸⁹Zr-AOD-Monocytes (⁸⁹Zr-MΦ) was tested by measuring the amount of ⁸⁹Zr released over a 48 h incubation period.

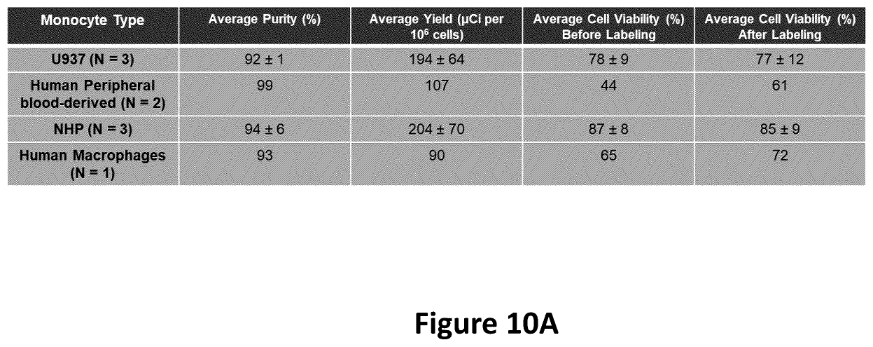

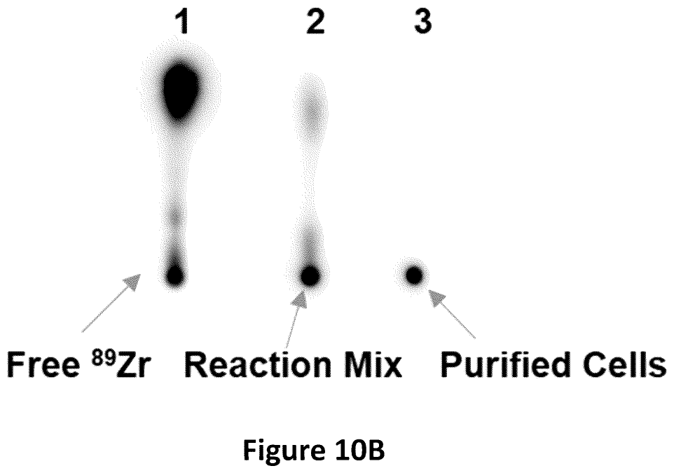

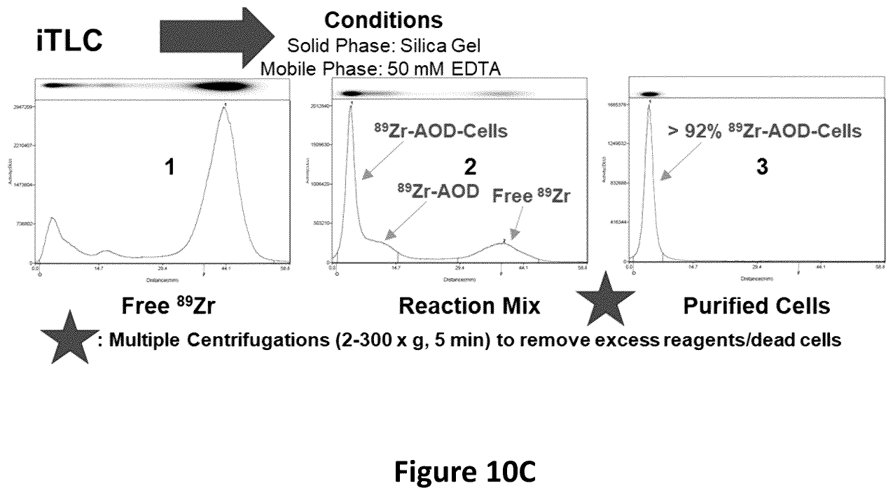

Results: ⁸⁹Zr-AOD-monocytes were successfully generated using the techniques described above. The labeling method resulted in significantly higher labeling yields than current direct radiolabeling methods and enabled the incorporation of higher amounts of radiotracer per cell, allowing for tracking fewer cells for longer periods in vivo (FIGS. 10A-10C).

FIG. 7B shows the expected peak of AOD molecular weight at 656.3574 as a result from method 1. Radiolabeled ⁸⁹Zr-MΦ on cell surface glycans show nearly 75-fold higher molar activity (˜75-500 Ci/10⁶ cells) compared to cell surface protein radiolabeling and other direct radiolabeling methods.

Because different cell types may exhibit varying levels of surface glycans, a more potent oxidation catalyst can be employed or alternatively (or in addition) cells can be supplemented with a sialic acid analog, such as 9-deoxy-9-N-carboxybenzaldehyde-NeuAc (BA-NeuAc). Additionally, although expected to be stable at physiological pH, if an oxime ligation shows poor stability, the Pictet-Spengler ligation can be used to yield hydrolytically stable conjugates (Kölmel and Kool 2017).

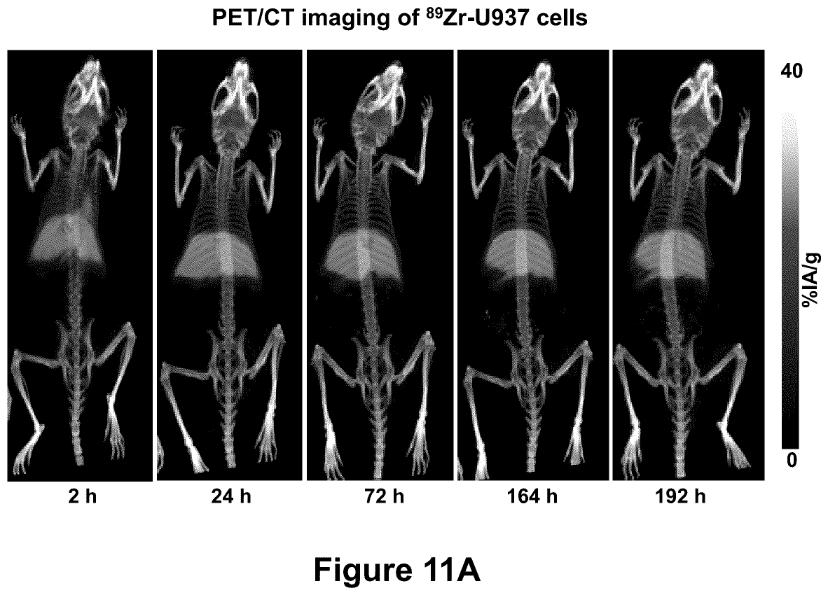

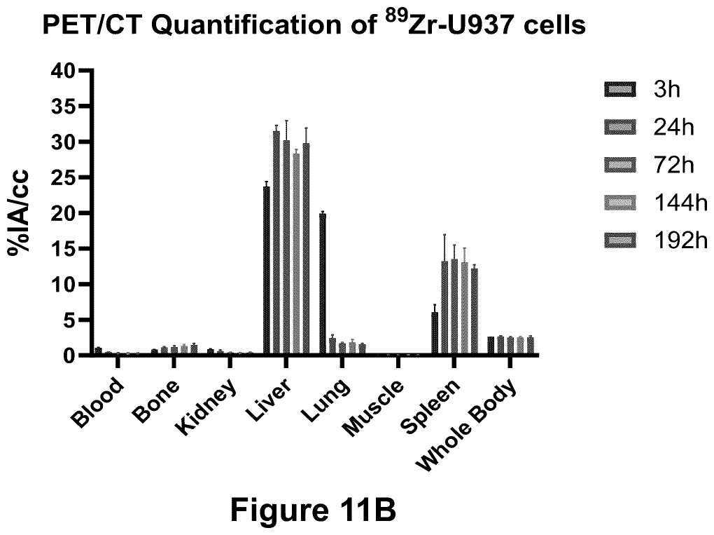

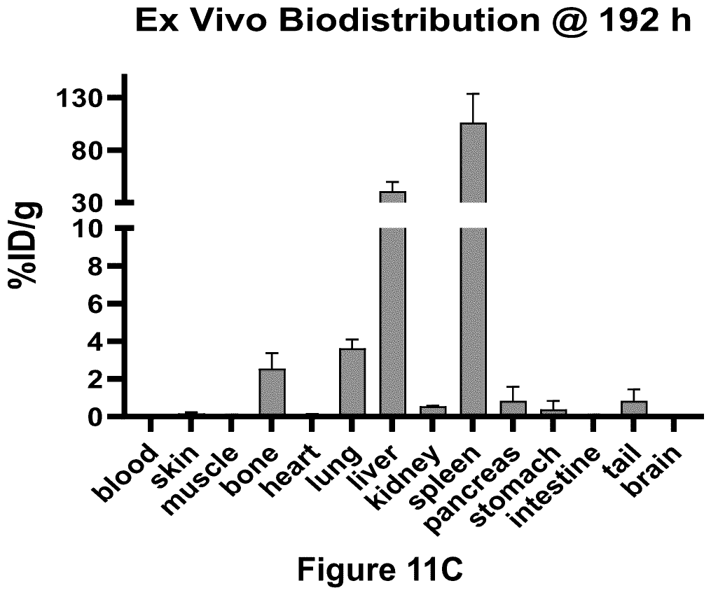

Overall, glycan oxidation with NaIO₄ (FIG. 8) had minimal effect on cell viability at the tested temperature and reaction times. Similarly, AOD conjugation and ⁸⁹Zr-labeling did not affect viability, which remained over 93% post-radiolabeling. Unprecedented radiochemical yields observed in U937 cells, ranging from 6.1-10.2 MBq per 10⁶ live cells, a 100-fold improvement over other methods, enabled tracking fewer cells (˜300,000 cells) with improved detectability. For in vivo PET/CT imaging, male NSG mice (N=3) were administered ⁸⁹Zr-U937 cells (3-5×10⁵; 5.6 MBq) or ⁸⁹Zr-PBMC (1-2×10⁵; 0.74 MBq) via tail vein injection and static PET/CT scans acquired at 3, 24, 72, 164, and 192 h post-injection (p.i.). Region-of-interest analysis and biodistribution studies were performed to quantify the labeled cells' longitudinal in vivo distribution and clearance. PET/CT images showed initial accretion of ⁸⁹Zr-U937 cells in the lungs (19.9±0.3% IA/g) and the liver (23.7±0.7% IA/g) at 3 h p.i., and their subsequent redistribution to the liver (31.5±0.8% IA/g) and spleen (13.5±2.0% IA/g) at later time points (FIG. 11A-11C). The low bone uptake and minimal whole-body radioactivity excretion were indicative of the excellent stability of the label in vivo. Similar distribution and retention were noted for ⁸⁹Zr-PBMC with prominent lung, liver, and spleen uptake.

### CONCLUSION

The results, taken together, showed unprecedented ⁸⁹Zr radiolabeling efficiencies with minimal impact on cell viability, affording improved imaging performance and hinting at the potential clinical value of this method for in vivo cell tracking.

### Example 2: Radiolabeling and Non-Invasive Cell Tracking of Human Peripheral Mononuclear Cells

Method: Peripheral blood mononuclear cells (PBMC) were isolated from human blood using Ficoll-Paque density centrifugation gradients. Conjugation and radiolabeling with PBMC were accomplished using the optimized methodologies in Example 1. For PET scans, NSG mice were injected ˜20 Ci (2.0-2.5×10¹ cells/injection)⁸⁹Zr-labeled PBMC and longitudinal PET/CT scans were acquired over an 8 day period. Ex vivo biodistribution was performed at day 8 to provide more accurate tissue distribution and confirm PET/CT.

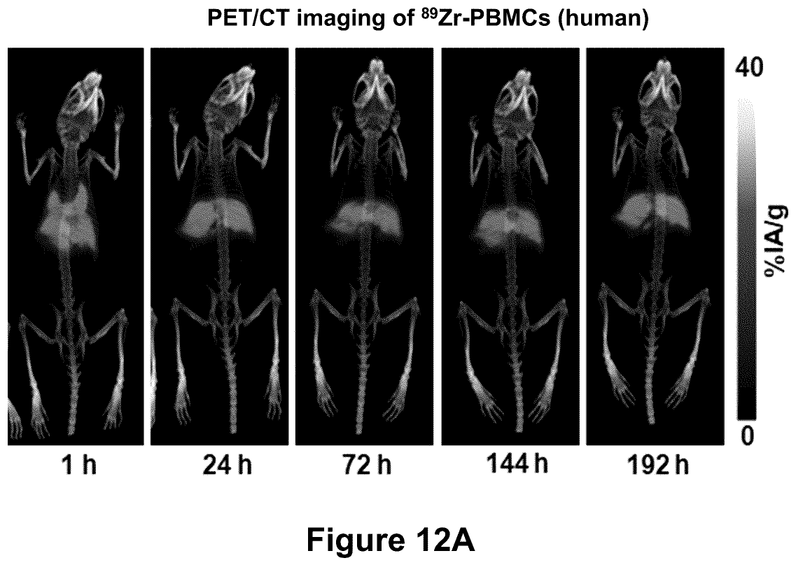

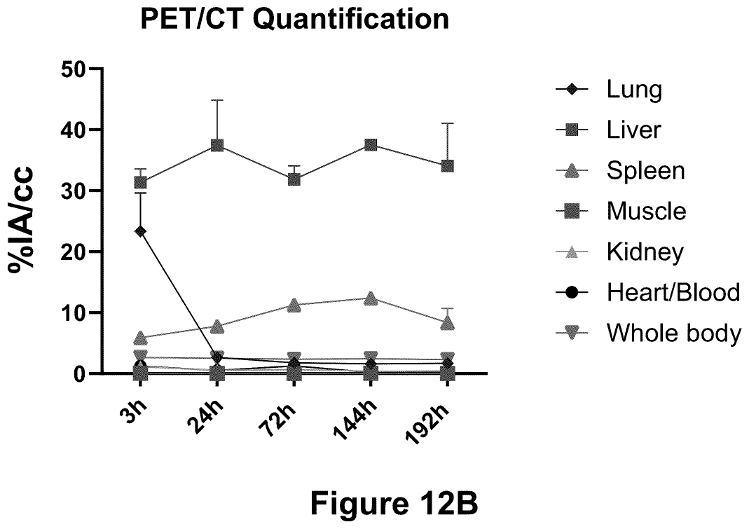

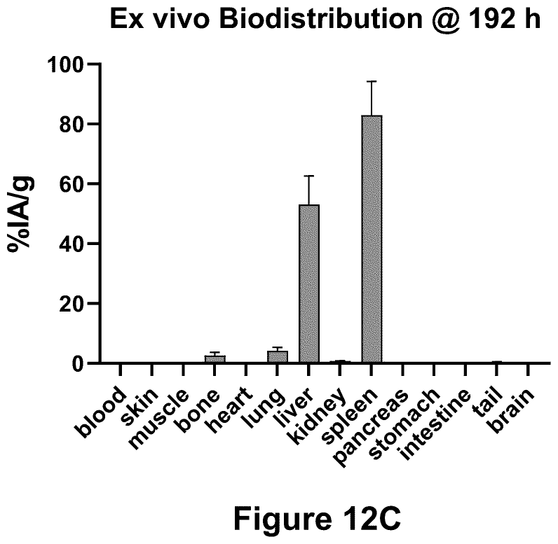

Results: Interestingly, the viability of PBMC increased from an initial 43% to 65% after radiolabeling, plausibly due to the removal of a significant portion of dead cells during the conjugation and radiolabeling process. Based on PET/CT imaging (FIGS. 12A and 12B) and biodistribution (FIG. 12C), injected cells initially lodged in the lungs and gradually migrated to the liver and spleen, following the general behavior observed for intravenously injected cells. Ex vivo biodistribution showed the highest uptake in the spleen followed by the liver. Again, whole-body uptake remained constant during the observation period.

### Example 3: Determining the Trafficking of iPSC Derived Macrophages from Two Different Cell Sources in by PET/CT

Introduction: The ability to determine the trafficking of infused therapeutic monocytes/macrophages to sites of disease and healthy tissues is instrumental to designing and predicting the success of these living therapeutics. Here, the in vivo biodistribution of systemically administered ⁸⁹Zr-labeled macrophages obtained from human and non-human primate induced pluripotent stem cells in mice by PET/CT is determined. It is contemplated that macrophages derived from bone marrow or iPSCs can be tracked noninvasively and that they will show a similar biodistribution in healthy mice. The successful development of a translationally relevant methodology for longitudinal PET/CT tracking of macrophages may enable the optimization of these cell therapies in the context of a disease model. It is contemplated that glycan-based direct radiolabeling of macrophages with ⁸⁹Zr having is both feasible and safe. Additionally, it is contemplated that iPSC and BM-derived macrophages have equivalent trafficking patterns and abilities to infiltrate tissues like the bone marrow or the brain. Given the similarities between monkeys and humans, both anatomically and physiologically, these results are of immediate relevance to inform the implementation of similar approaches in humans.

Method: Mauritius Cynomolgus Macaque (MCM) and human macrophages were differentiated from MCM iPSCs, as described previously (D'Souza, Maufort et al. 2016). To covalently conjugate AOD to the surface of living cells, reactive aldehyde groups were generated in the membrane glycans through periodate-mediated cleavage of vicinal diols in sialic acid. Initially, conditions to generate significant amounts of cell-surface aldehydes without compromising cell viability were optimized. To this end, 1-10×10⁶ iPSC-derived monocytes were incubated with increasing concentrations of NaIO₄ (1 mM) at 37° C., for 1 hour, with the presence of a catalyst (aniline). Cell viability was assessed for each reaction condition via trypan blue staining. For PET imaging, NSG mice were injected ˜25 μCi (0.15-2.5×10⁵ cells/injection) ⁸⁹Zr-labeled macrophages derived from NHP and Human iPSC. Longitudinal PET/CT scans were acquired over a 7 day period. Ex vivo biodistribution was performed at day 7 to provide more accurate tissue distribution and confirm PET/CT. Quantitative imaging and biodistribution data are reported as percent injected activity per gram of tissue (% IA/g).

Results: The feasibility and safety of the non-invasive tracking of monocytes from human and non-human primates by PET/CT was demonstrated. Additionally, labeling and scanning protocols are implemented that will serve as the base to further investigation using therapeutic monocytes in disease models. Similar trafficking patterns of monocytes derived from human or MCM iPSC were observed. Finally, imaging protocols are established that will serve as a reference for other studies investigating additional cell therapies in non-human primates and humans.

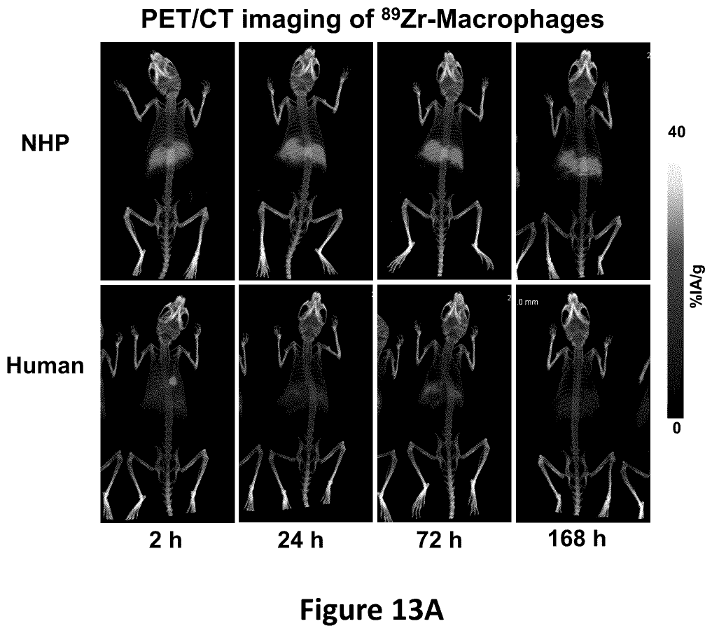

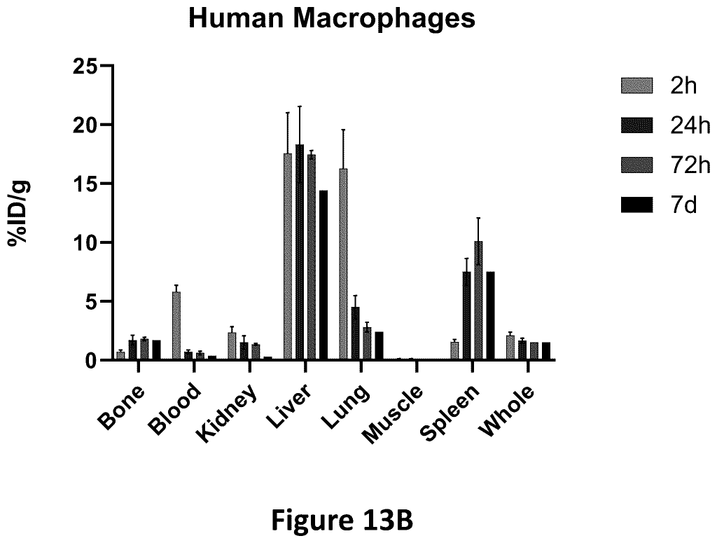

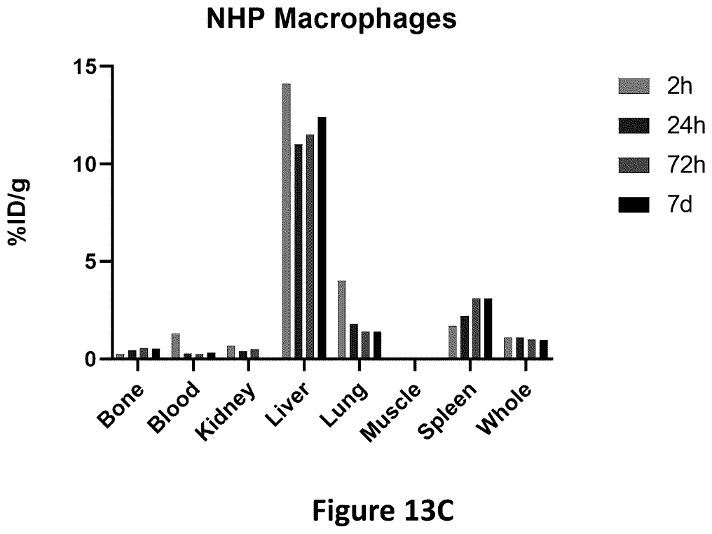

FIG. 13A shows acquired PET scans at 2, 24, 72, and 168 hours post-injection of the radiolabeled cells. The ⁸⁹Zr-MΦ initially homing to the lungs, heart, and liver. Heart and lung signals declined over time, indicating a significant redistribution of the cells from these organs into the liver and spleen. Quantitative analysis of the PET/CT images (FIGS. 13B & 13C) showed very similar patterns of distribution to the liver, lungs, and spleen for the humans and MCM-derived macrophages. Whole-body radioactivity retention demonstrated the poor excretion of the administered cells and the high stability of the radiolabel.

Because PET/CT signal to noise (SN) ratios can vary, if a low SN is seen, the number of administered cells can be increased to enhance PET image quality. Conversely, if high radiation doses to the animal are observed, the amount of label per cell (mCi/10\hat{ }6) and/or the number of administered cells can be reduced. In this example, as few as 15,000 radiolabeled cells were able to be imaged and tracked successfully.

### Example 4: In Vivo PET/CT Tracking of Human and Primate Monocytes Using Radiochemistry

Introduction: Monocytes are vital in the innate immune response and have been investigated as a cell-based therapy for multiple diseases. Understanding their in vivo biodistribution is essential for determining their therapeutic efficacy. Yet, current radiolabeling limitations, including low radiochemical yield and poor stability, hinder in vivo tracking methods using PET/CT. To combat these limitations, a radiochemical protocol was developed that directly conjugates a derivative of Deferoxamine (DFO), aminooxy-DFO (AOD), to oxidized cell-surface sialic acids, allowing increased radiochemical yield and stability while maintaining cell viability.

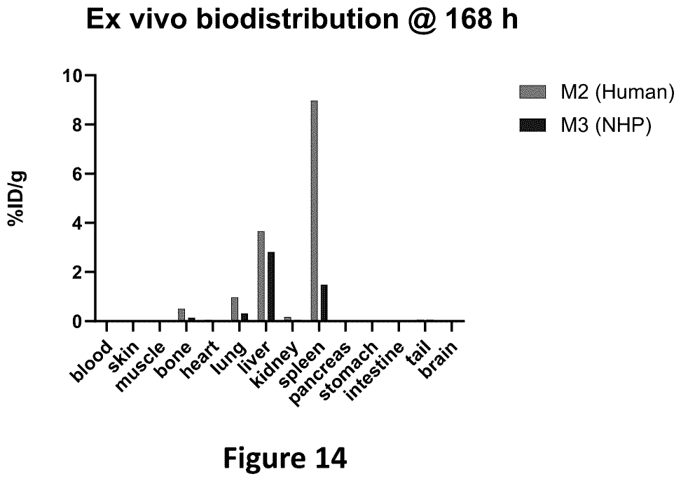

Method: Optimization of the sialic acid oxidation conditions, including temperature, [NaIO₄], and time, was performed using immortalized U937 monocytes. For radiolabeling studies, CD14+ monocytes obtained from human peripheral blood (HPB-Mos) or non-human primate induced pluripotent stem cells (iPSCs) (NHP-Mos) were suspended in 1 mL PBS (pH 7.4) and oxidized with NaIO₄ (1 mM) for 20 min to oxidize cell surface sialic acids. Upon washing and resuspension into 1 mL PBS (pH 6.5), the cells were incubated with AOD (200 μM) in aniline (10 mM) for 40 min. Once conjugated, the cells (2-5×10⁶) were radiolabeled with ⁸⁹Zr (33-37 MBq) for 40 min and subsequently centrifuged to remove unconjugated ⁸⁹Zr. Cell viability was measured at each step via trypan blue staining, and radiochemical yield and purity were determined via radiosensitive TLC. Radiolabeled ⁸⁹Zr-HPB-Mos (4.6×10⁵ cells, 3.27 MBq) or ⁸⁹Zr—NHP-Mos (1.3-1.5×10⁶ cells, 4.551-5.624 MBq) were intravenously administered into NSG mice (N=6) and PET/CT imaging was performed at 2, 24, 72, 144 and 192 h post-injection. Ex vivo biodistribution was carried out at 168h (FIG. 14). Biodistribution data was reported as percent injected activity per gram of tissue (% IA/g).

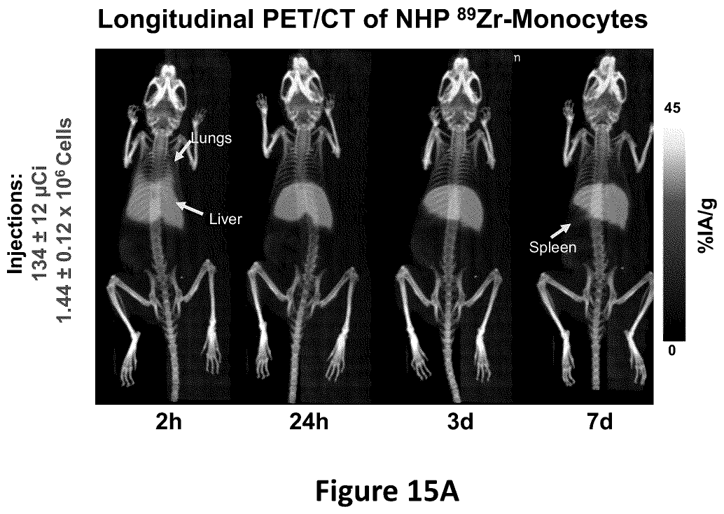

Results: Monocytes were efficiently radiolabeled with ⁸⁹Zr (4.3-7.1 MBq per 1×10⁶ live cells) with excellent purity (93-95%) and viability (70-75%) (FIGS. 10A-10C). Region-of-interest (ROI) analysis quantified ⁸⁹Zr-HPB-Mos in vivo biodistribution, indicating initial accumulation of the cells in the lungs (25.6±5.1% IA/g) and liver (39.7±2.8% IA/g) at 2h, followed by redistribution to the liver (41.5±3.8% IA/g) and spleen (6.85±0.5% IA/g) at 168h. Similarly, ROI analysis of the PET/CT imaging showed uptake of ⁸⁹Zr—NHP-Mos in the liver (42.5±6.5% IA/g) and lungs (21.9±10.2% IA/g) at 2h, followed by liver retention (47.3±8.4% IA/g), spleen accretion (10.7±0.96% IA/g) and lung clearance at 168h (FIGS. 13A-13C). The biodistribution study performed after the terminal PET/CT scan showed concordant uptake with the 192h scan in the liver (54.0±5.7 vs. 50.1±12.8% IA/g) and spleen (58.1±7.0 vs. 56.6±9.8% IA/g) for ⁸⁹Zr-HPB-Mos vs. ⁸⁹Zr—NHP-Mos, respectively (FIG. 13A and FIG. 15A). Overall, there was negligible normal tissue uptake and minimal whole-body radioactivity excretion, indicative of the excellent stability of the label in vivo.

Conclusion: These results proved that the ⁸⁹Zr radiolabeling efficiency and maintained cell viability enhanced PET/CT imaging performance by increasing the signal-to-noise ratio, thereby improving detectability and precise in vivo tracking of therapeutic monocytes. The versatility, consistency, and simplicity the protocol offers is evidenced by the tracking of multiple monocyte subtypes.

### Example 5: Annotating the Distribution of Stem Cell-Derived Macrophages in Non-Human Primates by PET/CT

Introduction: Cell therapies, such as therapeutic macrophages (Mϕ), are hampered by the limited availability of these cells at scale. Deriving macrophages from induced pluripotent stem cells (iPSC) addresses supply concerns, but whether iPSC-derived macrophages (iMϕ) cells traffic similarly to autologous cells in vivo remains unknown. Using innovative radiochemistry, iMϕ biodistribution were radiolabeled and tracked in non-human primates (NHP) by PET/CT.

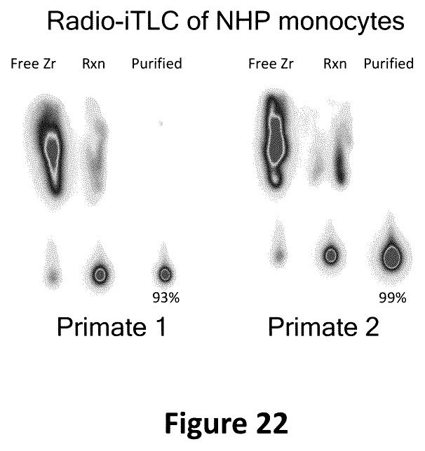

Materials and Methods: Monocyte radiolabeling with ⁸⁹Zr involved direct Deferoxamine (DFO) conjugation to oxidized cell-surface glycans using NaIO₄. The process was optimized using immortalized U937 monocytes. Oxidized cells (1-5×10⁶) were incubated with aminooxy-DFO (AOD: 200 μM) in aniline (10 mM), then radiolabeled with ⁸⁹Zr (33-37 MBq). Cell viability was measured at each step via trypan blue staining, and radiochemical yield and purity were determined via iTLC (FIG. 22). For imaging studies, human peripheral blood CD14+Mϕ(HPB-Mϕ) or NHP iPSCs (NHP-iMϕ) were radiolabeled, and [⁸⁹Zr]Zr-HPB-Mϕ(4.6×10⁵ cells, 3.3 MBq) or [⁸⁹Zr]Zr-NHP-Mϕ(1.3-1.5×10⁶ cells, 4.6-5.6 MBq) were injected IV in NSG mice (N=6) and serial PET/CT imaging was performed at 2, 24, 72, 144 and 192 h p.i. Mauritius macaques (N=2) were given [⁸⁹Zr]Zr-NHP-Mϕ11.1 MBq (˜2×10⁶) IV and scanned in a clinical PET/CT scanner at 0.5, 24, 72, and 168 h p.i. Region-of-interest (ROI) analysis data was reported as percent injected activity per gram (% IA/g) or standardized uptake values (SUVₘₑₐₙ).

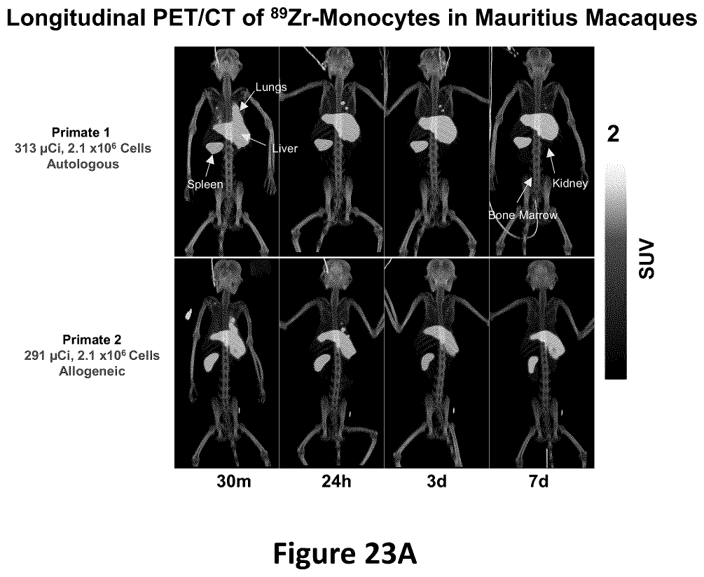

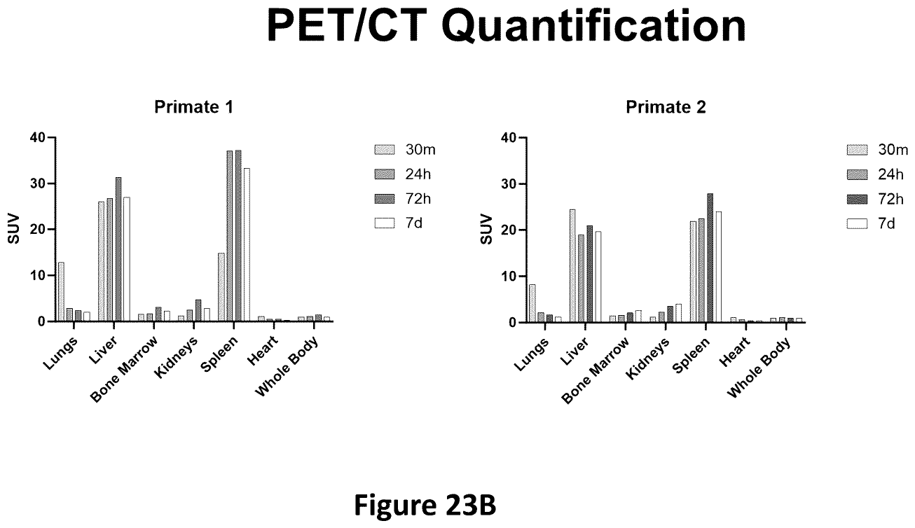

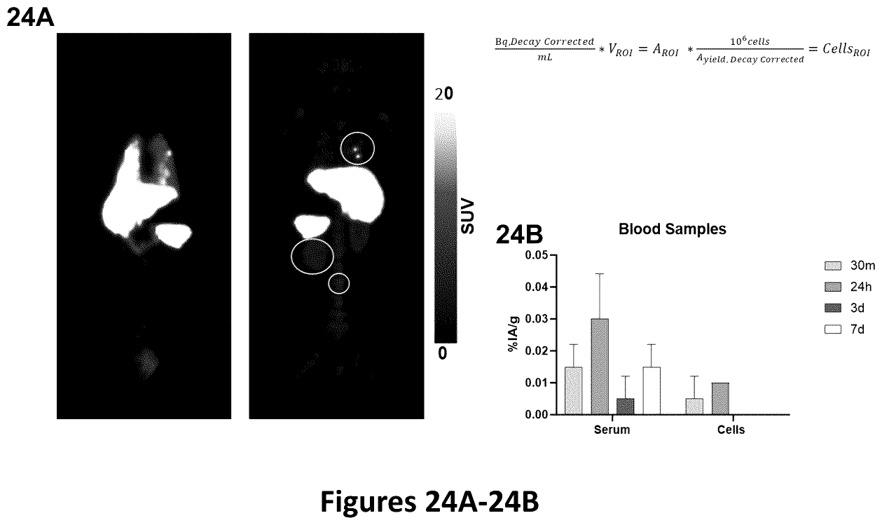

Results: Mϕ were efficiently radiolabeled with ⁸⁹Zr (4.3-7.1 MBq per 1×10⁶ cells) with excellent purity (93-95%) and viability (>75%). Mice administered [⁸⁹Zr]Zr-NHP-iMϕ showed similar in vivo biodistribution with initial cell accumulation in the lungs (21.9±10.2% IA/g) and liver (42.5±6.5% IA/g) at 2h followed by redistribution to the liver (41.5±3.8% IA/g) and spleen (10.7±0.96% IA/g) at 168h. Similarly, [⁸⁹Zr]Zr-NHP-iMϕ distribution in NHP displayed initially elevated lung (10.5±3.3 SUVₘₑₐₙ), liver (25.2±1.1 SUVₘₑₐₙ), and spleen (18.3±5.0 SUVₘₑₐₙ) accumulation at 30 min p.i. Later time points revealed a steady decline in lung activity and stable signal in the liver and spleen at day 7 (FIGS. 23A and 23B). The high labeling efficiency of [⁸⁹Zr]Zr-NHP-iMϕ, negligible normal tissue uptake, and minimal whole-body radioactivity excretion in both mice and NHP indicated cell persistence and excellent radiolabel stability in vivo, enabling the detection and monitoring of small cell clusters (FIGS. 24A-24B).

Conclusion: The ⁸⁹Zr radiolabeling protocol showed unprecedented efficiency and biocompatibility, allowing in vivo tracking of multiple macrophage subtypes. PET/CT studies in Mauritius macaques evidenced the clinical utility of the tracking methodology and the potential of iPSC-derived therapeutic Mb.

### Example 6: Radiolabeling and In Vivo Cell Tracking of ⁸⁹Zr-Labeled Neutrophils and T Cells

Introduction: The ability to determine the trafficking of infused therapeutic neutrophils/T cells to sites of disease and healthy tissues is instrumental to designing and predicting the success of these living therapeutics. Here, it was determined that the in vivo biodistribution of systemically administered ⁸⁹Zr-labeled neutrophils or T cells obtained from either non-human primate induced pluripotent stem cells (neutrophils) or as an immortalized acute leukemia human cell line (T cells) in mice by PET/CT. The successful development of a translationally relevant methodology for longitudinal PET/CT tracking of potentially therapeutic neutrophils or T cells, including those adapted with chimeric antigen receptors (CAR-T cells, CAR-neutrophils) may enable the optimization of these cell therapies in the context of a disease model. Finally, it is contemplated that glycan-based direct radiolabeling of neutrophils or T cells with ⁸⁹Zr having is both feasible and safe.

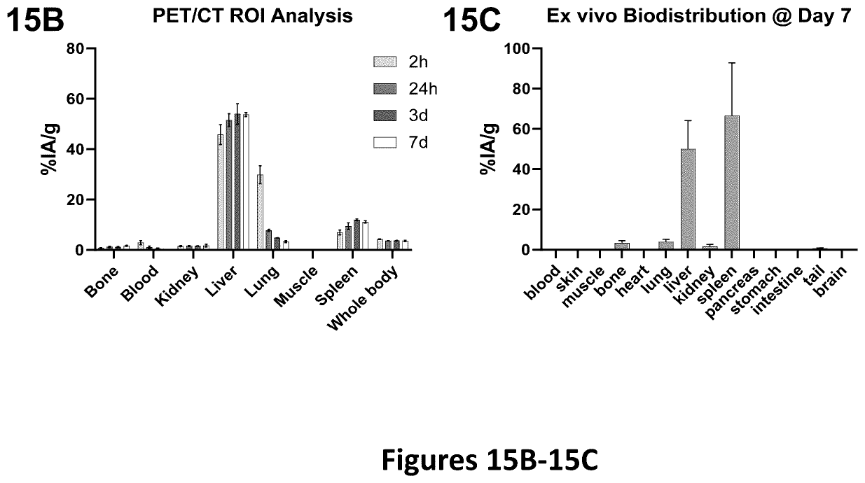

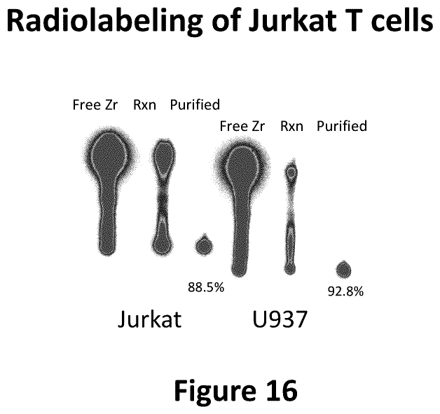

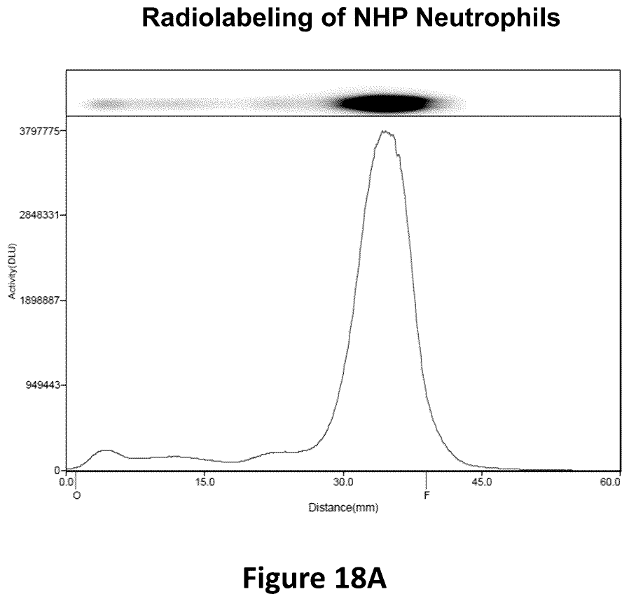

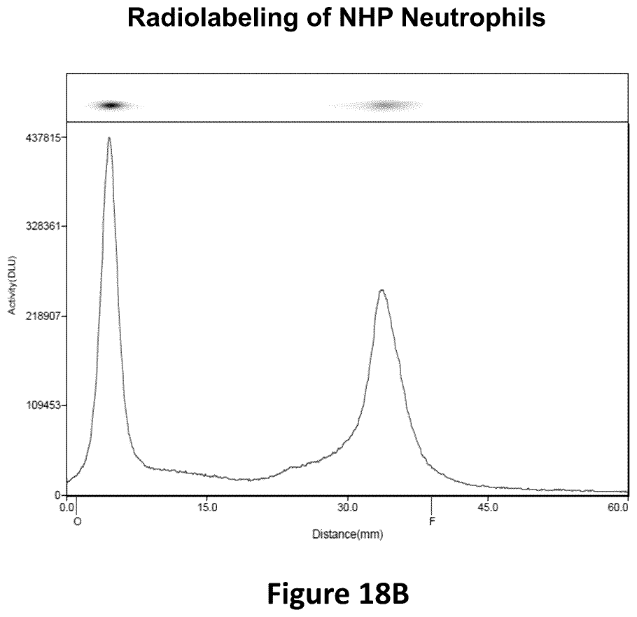

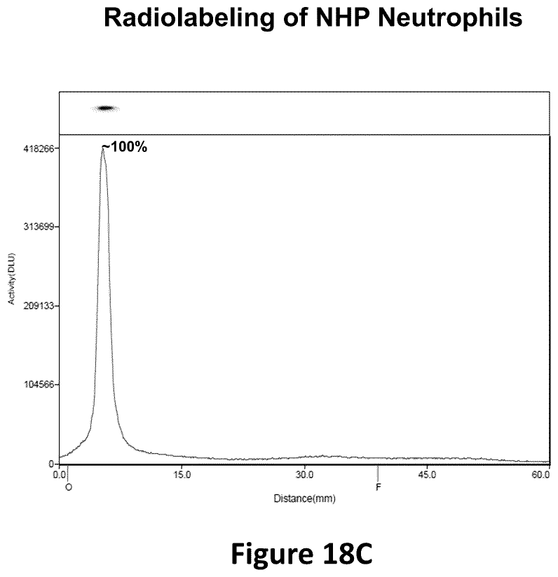

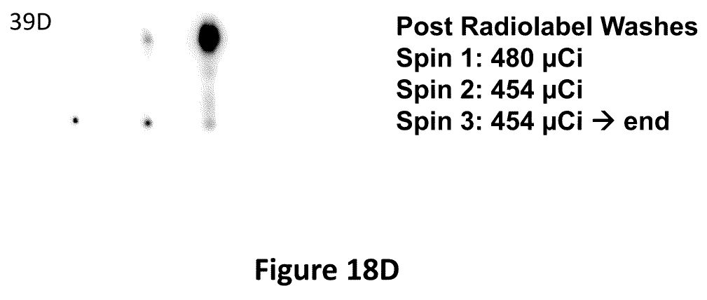

Materials and Methods: Neutrophil or Jurkat T cell radiolabeling with ⁸⁹Zr involved direct Deferoxamine (DFO) conjugation to oxidized cell-surface glycans using NaIO₄. The process was previously optimized using immortalized U937 monocytes (See Example 1). Oxidized cells (1-1.5×10⁷) were incubated with aminooxy-DFO (AOD: 200 M) in aniline (10 mM), then radiolabeled with ⁸⁹Zr (1.5 mCi). Cell viability was measured at each step via trypan blue staining, and radiochemical yield and purity were determined via iTLC (FIGS. 16, 18A-18D). For imaging studies, iPSC-derived non-human primate neutrophils (NHP-neutrophils) or Jurkat T Cells were radiolabeled, and [⁸⁹Zr]Zr—NHP-Neutrophils (1.2-1.46×10⁶ cells, 77-93 μCi) or [⁸⁹Zr]Zr-Jurkat T Cells (5.2×10⁶ cells, 69 μCi) were injected IV in NSG mice (N=3 for NHP-Neutrophils, N=1 for Jurkat T Cells) and serial PET/CT imaging was performed at 2, 24, 72, 144 and 192 h p.i. Region-of-interest (ROI) analysis data was reported as percent injected activity per gram (% IA/g).

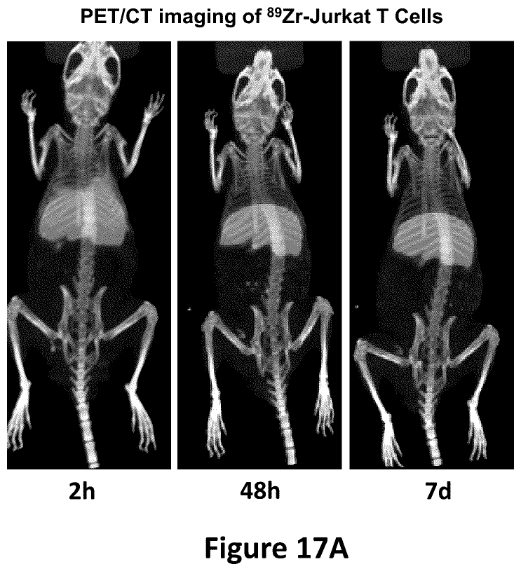

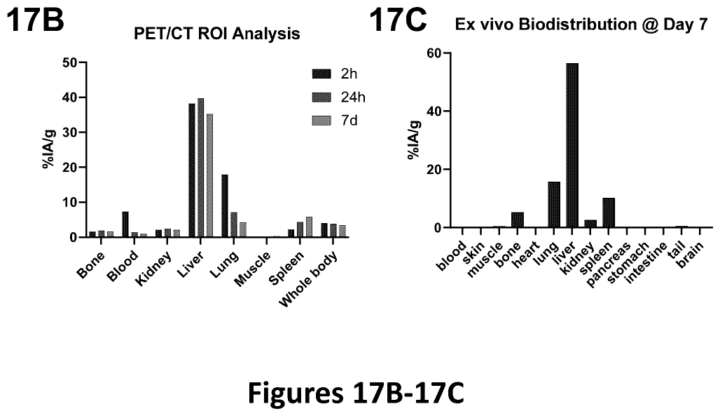

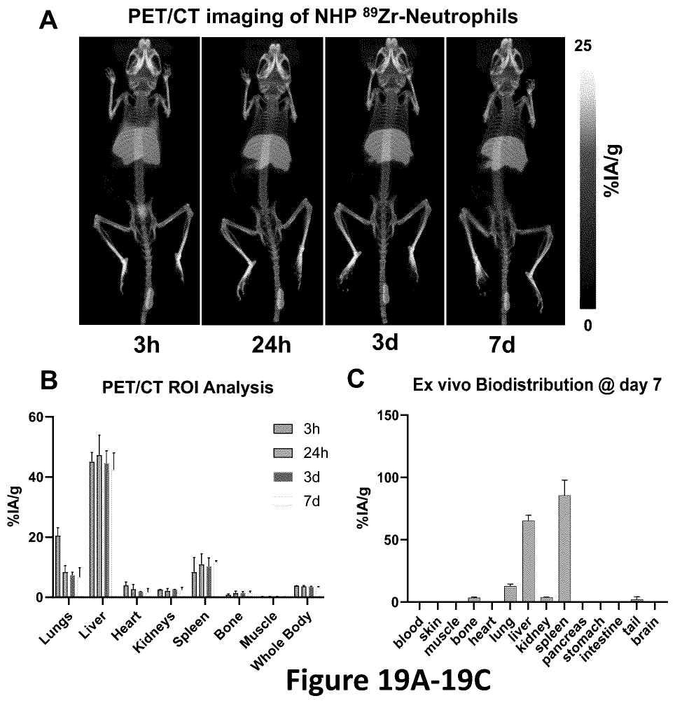

Results: NHP-Neutrophils and Jurkat T cells were efficiently radiolabeled with ⁸⁹Zr (64 μCi per 1×10⁶ NHP-Neutrophils, 133 μCi per 1×10⁶ Jurkat T Cells) with excellent purity (88-100%) and viability (76-91%). NSG Mice administered [⁸⁹Zr]Zr—NHP-Neutrophils showed similar in vivo biodistribution to NHP-monocytes, with initial cell accumulation in the lungs (20.5±2.6% IA/g) and liver (45.0±3.2% IA/g) at 2h followed by redistribution to the liver (42.4±5.7% IA/g) and spleen (11.7±0.5% IA/g) at 168h (FIGS. 19A-19C). Similarly, an NSG mouse administered [⁸⁹Zr]Zr-Jurkat T Cells again showed similar biodistribution to NHP-monocytes and NHP-neutrophils, with initial cell signal in the lungs (17.9% IA/g) and liver (38.2% IA/g) at 2h followed my redistribution to the liver (35.2% IA/g), lungs, (4.2% IA/g) and spleen (5.8% IA/g) (FIGS. 17A-17C).

Conclusion: The feasibility of the novel ⁸⁹Zr radiolabeling strategy in neutrophils and T cells was successfully validated. Compared to labeled monocytes, radiolabeling of these two cell types corroborated the unprecedented efficiency, biocompatibility, and robustness of the methodology.

### Example 7: Clodronate-Mediated Macrophage Depletion Mobilizes Labeled ⁸⁹Zr-MΦ in Vivo

Introduction: Understanding cell fate (viability, maintained function) is essential to designing and optimizing cellular therapies. Methods to radiolabel and track the distribution of these cell noninvasively can enable the study of their in vivo distribution using PET imaging. A significantly improved methodology was developed to radiolabel therapeutic cells including macrophages; however, the viability of the labeled cell after injection remains of concern. Herein, the distribution of ⁸⁹Zr-MΦ were tested in vivo via treatment with clodronate, a toxic undigestible metabolite than can be encapsulated in liposomes and selectively delivered to MΦ by phagocytosis, leading to apoptosis.

The hypothesis was that, “pre-treating” NSG mice with clodronate liposomes allows for new potential “reservoirs” of administered ⁸⁹Zr-MΦ that replace the killed resident MΦ, and that the administration of clodronate liposomes after ⁸⁹Zr-MΦ can alter the observed PET signal distribution, either due to the killing of ⁸⁹Zr-MΦ or resident MΦ. Using ⁸⁹Zr-labeled U937 monocytes, these conditions were tested in NSG mice and observed the changes in ⁸⁹Zr-MΦ biodistribution indicative of their phagocytic activity.

Materials and Methods: U937 monocytes were radiolabeled with ⁸⁹Zr using previously described methods (FIGS. 6, 9A-9D, 10A-10C). Clodronate liposomes were purchased (Liposoma BV, Amsterdam, Netherlands) and administered (100 μL of 5 mg/mL solution per 10 g body weight) to NSG mice 24 h pre- or post-administration of [⁸⁹Zr]Zr-MΦs. [⁸⁹Zr]Zr-MΦ(1.0-1.2×10⁶ cells, 28-34 μCi were injected IV in NSG mice (N=3) and serial PET/CT imaging was performed at 2, 24, 72, 144 and 192 h p.i. Region-of-interest (ROI) analysis data was reported as percent injected activity per gram (% IA/g).

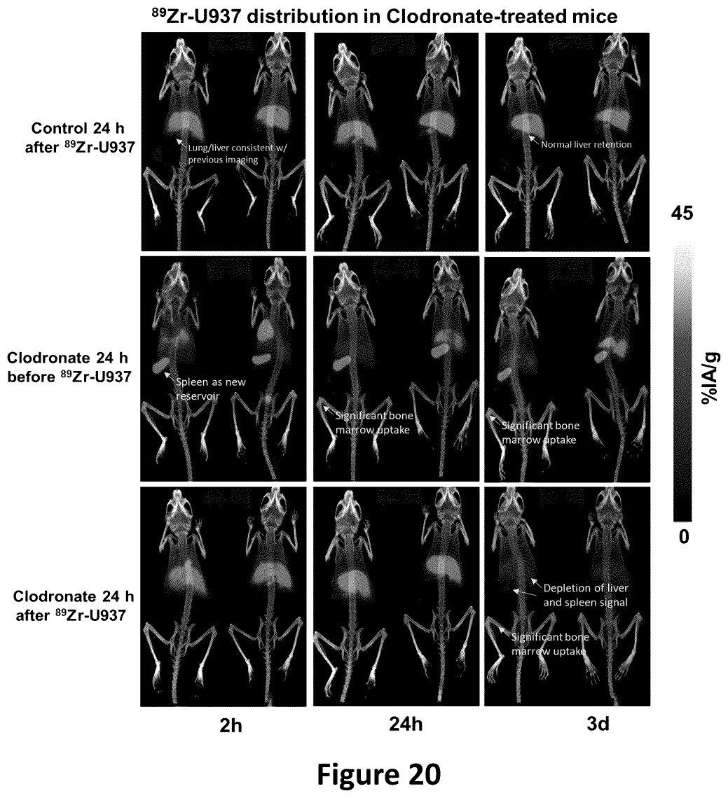

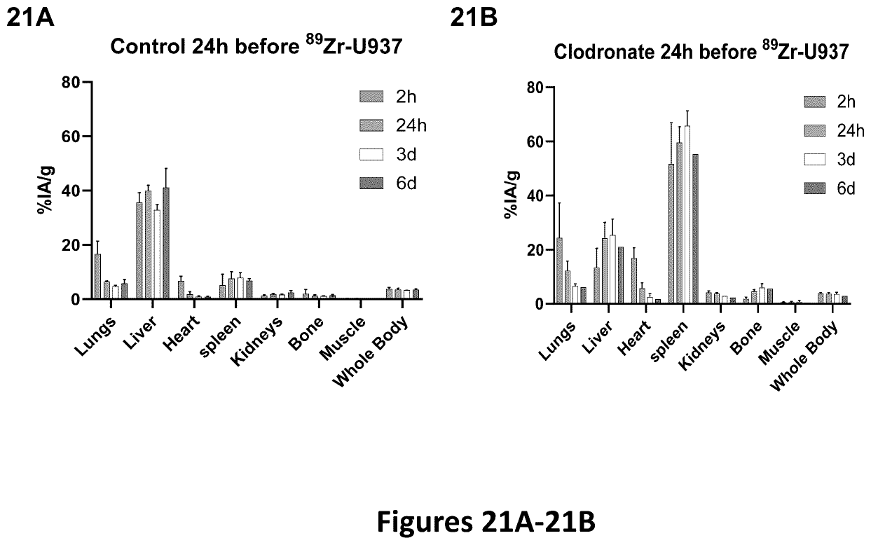

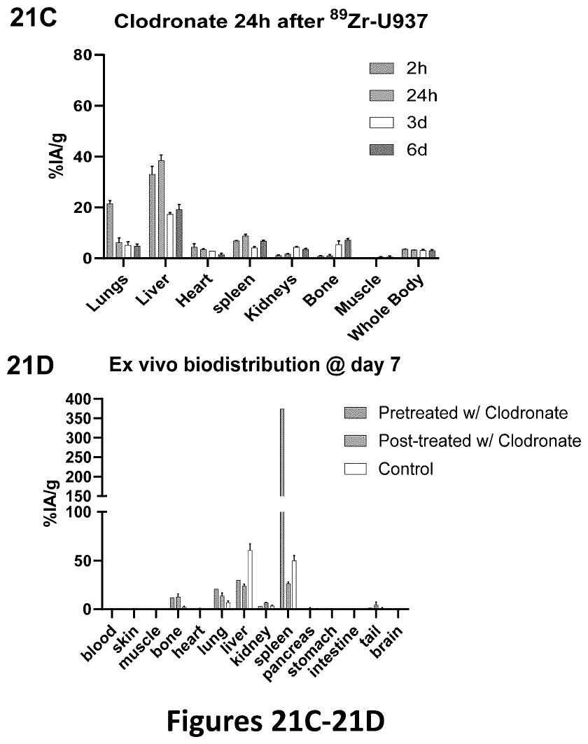

Results: MΦ were efficiently radiolabeled with ⁸⁹Zr (30 μCi per 1×10⁶MΦ) with excellent purity (88%) and viability (83%). NSG Mice administered [⁸⁹Zr]Zr-MΦ and PBS-control liposomes 24h post-administration of ⁸⁹Zr-MΦ showed similar in vivo biodistribution to previous U937 monocyte imaging (Example 1), with initial cell accumulation in the lungs (16.6±4.8% IA/g) and liver (35.5±3.7% IA/g) at 2h followed by redistribution to the liver (41.1±7.1% IA/g) and spleen (6.7±0.8% IA/g) at 168h (FIGS. 20, 21A-21D). However, NSG mice administered [⁸⁹Zr]Zr-MΦ and clodronate liposomes 24h post-administration of ⁸⁹Zr-MΦ showed a markedly different biodistribution at 3 and 7 days post-administration of ⁸⁹Zr-MΦ, with reduced liver (17.4±0.3% IA/g) and markedly higher bone marrow uptake (5.4±1.4% IA/g) at 3 days that persisted to 7 days. Finally, NSG Mice administered [⁸⁹Zr]Zr-MΦ and clodronate liposomes 24 h prior to administration of ⁸⁹Zr-MΦ showed significantly higher spleen (51.6±15.3% IA/g) and lungs (24.4±12.9% IA/g) uptake at 2h, and redistribution mainly the spleen at 7 days (55.3% IA/g). Interestingly, the liver uptake was lower than previously observed at 2h hours (18.7±2.3% IA/g), a number that persisted in a 7 days (21% IA/g) (FIGS. 20, 21A-21D).

Conclusion: These results indicate that the biodistribution of ⁸⁹Zr-MΦ can be modulated based on the administration of clodronate liposome in vivo. This study indicates the viability and phagocytic function of the injected ⁸⁹Zr-MΦ, corroborating the biocompatibility of the radiolabeling methodology.

The embodiments illustratively described herein suitably can be practiced in the absence of any element or elements, limitation or limitations that are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the embodiments claimed. Thus, it should be understood that although the present description has been specifically disclosed by embodiments, optional features, modification and variation of the concepts herein disclosed may be resorted to by those skilled in the art, and that such modifications and variations are considered to be within the scope of these embodiments as defined by the description and the appended claims. Although some aspects of the present disclosure can be identified herein as particularly advantageous, it is contemplated that the present disclosure is not limited to these particular aspects of the disclosure.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein.

1. A method of labeling a cell for in vivo tracking, comprising:
(a) generating an aldehyde group on a cell surface glycan of a cell via reaction with an oxidizing agent; and
(b) reacting the aldehyde group on the cell surface glycan with an aldehyde-reactive bifunctional chelator,
wherein the bifunctional chelator reacts with the aldehyde group on the cell surface glycan to ligate the bifunctional chelator to the cell surface glycan to form a labeled cell.

2. The method of claim 1, wherein the bifunctional chelator is generated by conjugating an aldehyde/ketone-reactive group to a chelator.

3. The method of claim 2, wherein the aldehyde/ketone-reactive group is an aminooxy group.

4. The method of claim 2, wherein the aldehyde/ketone-reactive group is a hydrazine group.

5. The method of claim 2, wherein the chelator is deferoxamine.

6. The method of claim 2, wherein the chelator is 1,4,7,10-tetraazacyclododecane-1,4,7-triacetic acid (DO3A) or one of its derivatives; 1,4,7-triazacyclononane-1,4-diacetic acid (NODA) or one of its derivatives; 1,4,7-triazacyclononane-1,4,7-triacetic acid (NOTA) or one of its derivatives; 1,4,7,10-tetraazacyclododecane-1,4,7,10-tetraacetic acid (DOTA) or one of its derivatives; 1,4,7-triazacyclononane,1-glutaric acid-4,7-diacetic acid (NODAGA) or one of its derivatives; 1,4,7,10-tetraazacyclodecane,1-glutaric acid-4,7,10-triacetic acid (DOTAGA) or one of its derivatives; 1,4,8,11-tetraazacyclotetradecane-1,4,8,11-tetraacetic acid (TETA) or one of its derivatives; 1,4,8,11-tetraazabicyclo[6.6.2]hexadecane-4,11-diacetic acid (CB-TE2A) or one of its derivatives; diethylene triamine pentaacetic acid (DTPA), or its diester, or one of its derivatives; 2-cyclohexyl diethylene triamine pentaacetic acid (CHX-A″-DTPA) or one of its derivatives; 2-(4-Isothicyanatobenzyl)-1,2,7,10,13-hexaazacyclooctadecane-1,4,7,10,13,16-hexaacetic acid (HEHA) or one of its derivatives; deforoxamine (DFO) or one of its derivatives; 1,2-[[6-carboxypyridin-2-yl]methylamino]ethane (H2dedpa) or one of its derivatives; HOPO or one of its derivatives; MACROPA or one of its derivatives; a CROWN ether or one of its derivatives; or DADA or one of its derivatives.

7. The method of claim 3, wherein the bifunctional chelator is an aminooxy-deferoxamine.

8. The method of claim 1, wherein the oxidizing agent is NaIO₄.

9. (canceled)

10. The method of claim 1 further comprising conjugating a radionuclide to the bifunctional chelator.

11. (canceled)

12. The method of claim 10, wherein the radionuclide is ⁸⁹Zr, ⁶⁴Cu, ⁶¹Cu, ⁵²Mn, ⁵¹Mn, ⁸⁶Y, ¹¹¹In, ¹⁷⁷Lu, ¹⁵³Sm, ⁹⁹mTc, ⁶⁸Ga, ⁶⁷Ga, ⁶⁶Ga, ^{44/43}Sc, ⁴⁵Ti, ⁵⁵Co, ¹⁸F, ¹⁶¹Tb, or ¹⁵²Tb.

13. The method of claim 1, wherein the cell is a macrophage, a T-cell, a B-cell, a natural killer cell, a monocyte, a granulocyte, a dendritic cell, a neutrophil, a fibroblast, a mesenchymal stromal cell, or a peripheral blood mononuclear cell.

14. A method of generating a population of radiolabeled cells for in vivo tracking, comprising:
(a) generating aldehyde groups on cell surface glycans of a population of cells via reaction with an oxidizing agent;
(b) reacting the aldehyde groups on the cell surface glycans with an aldehyde-reactive bifunctional chelator to ligate the bifunctional chelator to the cell surface glycans; and
(c) conjugating a radionuclide to the bifunctional chelator to generate a radionuclide labeled cell population.

15. The method of claim 14, wherein viability of the cell population is greater than about 90% after step (a).

16. A method of tracking and imaging a radiolabeled cell in vivo, comprising:
(a) administering the radionuclide labeled cell of claim 14 to a subject; and
(b) tracking and imaging the radiolabeled cell in the subject by PET/CT or SPECT/CT or Planar scan of the subject.

17. The method of claim 16, wherein the radionuclide labeled cell is administered intravenously, intraperitoneally, subcutaneously, intrathecally, or intracranially.

18. A radionuclide labeled cell, comprising:
a cell surface glycan ligated with a bifunctional chelator,
wherein a radionuclide is conjugated to the bifunctional chelator.

19. The radionuclide labeled cell of claim 18, wherein the cell surface glycan is a sialic acid.

20. The radionuclide labeled cell of claim 18, wherein the bifunctional chelator comprises an aminooxy group or a hydrazine group conjugated to a chelator.

21. The radionuclide labeled cell of claim 18, wherein the chelator is deferoxamine, 1,4,7,10-tetraazacyclododecane-1,4,7-triacetic acid (DO3A) or one of its derivatives; 1,4,7-triazacyclononane-1,4-diacetic acid (NODA) or one of its derivatives; 1,4,7-triazacyclononane-1,4,7-triacetic acid (NOTA) or one of its derivatives; 1,4,7,10-tetraazacyclododecane-1,4,7,10-tetraacetic acid (DOTA) or one of its derivatives; 1,4,7-triazacyclononane,1-glutaric acid-4,7-diacetic acid (NODAGA) or one of its derivatives; 1,4,7,10-tetraazacyclodecane,1-glutaric acid-4,7,10-triacetic acid (DOTAGA) or one of its derivatives; 1,4,8,11-tetraazacyclotetradecane-1,4,8,11-tetraacetic acid (TETA) or one of its derivatives; 1,4,8,11-tetraazabicyclo[6.6.2]hexadecane-4,11-diacetic acid (CB-TE2A) or one of its derivatives; diethylene triamine pentaacetic acid (DTPA), or its diester, or one of its derivatives; 2-cyclohexyl diethylene triamine pentaacetic acid (CHX-A″-DTPA) or one of its derivatives; 2-(4-Isothicyanatobenzyl)-1,2,7,10,13-hexaazacyclooctadecane-1,4,7,10,13,16-hexaacetic acid (HEHA) or one of its derivatives; deforoxamine (DFO) or one of its derivatives; 1,2-[[6-carboxypyridin-2-yl]methylamino]ethane (H2dedpa) or one of its derivatives; HOPO or one of its derivatives; MACROPA or one of its derivatives; a CROWN ether or one of its derivatives; or DADA or one of its derivatives.

22. The radionuclide labeled cell of claim 18, wherein the radionuclide is ⁸⁹Zr, ⁶⁴Cu, ⁶¹Cu, ⁵²Mn, ⁵¹Mn, ⁸⁶Y, ¹¹¹In, ¹⁷⁷Lu, ¹⁵³Sm, ⁹⁹mTc, ⁶⁸Ga, ⁶⁷Ga, ⁶⁶Ga, ^{44/43}Sc, ⁴⁵Ti, ⁵⁵Co, ¹⁸F, ¹⁶¹Tb, or ¹⁵²Tb.
